(19) United States
(12) Patent Application Publication
(10) Pub. No.: US 2026/0009070 A1
(43) Pub. Date: Jan. 8, 2026

(54) LIBRARY PREPARATION AND ANALYTICAL METHODS FOR PRESERVING TOPOLOGICAL INFORMATION OF CELL-FREE DNA

(71) Applicants: Foundation Medicine, Inc., Boston, MA (US); Roche Sequencing Solutions, Inc., Pleasanton, CA (US)

(72) Inventors: Cassandra BURKE, Burlington, MA (US); Benjamin MCNALLY, Westwood, MA (US); David PENKLER, Santa Clara, CA (US)

(73) Assignees: Foundation Medicine, Inc., Boston, MA (US); Roche Sequencing Solutions, Inc., Pleasanton, CA (US)

(21) Appl. No.: 19/125,204

(22) PCT Filed: Dec. 15, 2023
(86) PCT No.: PCT/US2023/084292

§ 371 Date: Apr. 28, 2025

Related U.S. Application Data
(60) Provisional application No. 63/433,348, filed on Dec. 16, 2022.

(51) Int. Cl. C12Q 1/6869; C12Q 1/6886

(52) U.S. Cl. CPC C12Q 1/6869; C12Q 1/6886; C12Q 2600/156

(57) ABSTRACT

Methods and systems for constructing cfDNA sequence libraries, including methods and systems for sequencing 5′ and/or 3′ cfDNA overhangs to identify overhang length and sequence topology data are described herein. The method can comprise, for example, the use of the cfDNA topology data to generate cfDNA overhang sequence libraries.

### CROSS-REFERENCE TO RELATED APPLICATIONS

This application claims the priority benefit of U.S. Provisional Patent Application No. 63/433,348, filed Dec. 16, 2022, the entire contents of which are incorporated herein by reference for all purposes.

### FIELD OF THE INVENTION

Disclosed herein are methods and systems for determining and analyzing a DNA duplex, such as cell-free DNA (cfDNA), topological information and constructing sequencing libraries to obtain such topological information. Certain aspects of the disclosure relate more specifically to methods and systems for constructing DNA libraries for determining and/or analyzing the 5′ and 3′ overhangs from the DNA duplex (e.g., cfDNA).

### BACKGROUND

Cell-free DNA (cfDNA) molecules are free-floating double stranded DNA molecules (dsDNA or duplex DNA) found in the blood stream, typically as the result of cell apoptosis or necrosis, particularly in the context of disease, such as cancer. These degraded linear DNA fragments are often approximately 50-300 base pairs in length. Most commonly, cfDNA is assayed for cancer screening at early stages in disease progression by analyzing the cfDNA sequences to identify cancer-associated mutations.

Native cfDNA often have DNA ends where either the 5′ or 3′ end overhangs the complimentary strand, thereby resulting in jagged ends. Standard DNA sequencing methods are only capable of sequencing DNA with blunt ends, so traditional sequencing techniques involve the removal of these native topologies during the end repair step of traditional double stranded library construction. As a result, all information about any jagged end lengths and sequences, native end sequences, gaps, and nicks that were present in the cfDNA and the opportunity to identify any possible relevance of this information for cancer diagnosis and cancer care are lost.

### SUMMARY OF THE INVENTION

Described herein are methods for determining a cell-free DNA topology, and methods for making a nucleic acid construct for determining a cell-free DNA topology. Also described are methods of detecting diseased based, at least in part, on the determined cell-free DNA topology.

In some implementations, a method for determining cell-free DNA topology includes: extending a 3′ end of a first strand of a cell-free DNA duplex with inosine bases to fill a 5′ overhang of a second strand of the cell-free DNA duplex; attaching a sequencing adapter to the cell-free DNA duplex; sequencing the first strand of the cell-free DNA duplex to generate a first sequence read; determining, by one or more processors, one or more bases in the first sequence read to be soft-clipped; soft clipping, by the one or more processors, bases corresponding to a 3′ inosine extension of the first strand of the cell-free DNA duplex from the first sequence read; and determining, by the one or more processors, a length or sequence of the 5′ overhang of the second strand of the cell-free DNA duplex based on the soft clipping of the 3′ inosine extension of the first strand of the cell-free DNA duplex. The method may further include detecting, by the one or more processors, a presence or absence of a disease (such as cancer) based on the length or sequence of the 5′ overhang of the second strand of the cell-free DNA duplex.

The method for determining the cell-free DNA topology may further include attaching a second sequencing adapter to a 3′ overhang of the second strand of the cell-free DNA duplex; extending a 3′ end of the second sequencing adapter with inosine bases to fill the 3′ overhang of the second strand of the cell-free DNA duplex; attaching a 3′ inosine-extended end of the second sequencing adapter to a 5′ end of the first strand of cell-free DNA duplex, wherein the attached 3′ inosine-extended end provides a 5′ inosine extension of the first strand cell-free DNA duplex; determining, by one or more processors, one or more bases attached to the 5′ end of the first sequence read to be soft-clipped; soft clipping, by the one or more processors, bases corresponding to the 5′ inosine extension of the first strand of the cell-free DNA duplex from the first sequence read; and determining, by the one or more processors, a length or sequence of the 3′ overhang of the second strand of the cell-free DNA duplex based on the soft clipping of the 5′ inosine extension of the first strand of cell-free DNA duplex. The method may further include, in some embodiments, detecting, by the one or more processors, a presence or absence of a disease based on the length or sequence of 3′ overhang of the second strand of the cell-free DNA duplex. In some embodiments, attaching the second sequencing adapter to the 3′ overhang of the second strand of the cell-free DNA duplex comprises: extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and attaching the second sequencing adapter to the 3′ extension of the second strand of the cell-free DNA duplex. In some embodiments, the 3′ overhang of the second strand of the cell-free DNA duplex is extended using nucleotide bases of the same base type.

In some embodiments of the above methods, determining the one or more bases in the first sequence read to be soft-clipped comprises aligning the first sequence read to a reference sequence and identifying an unaligned portion of the first sequence read.

In some embodiments, the method for determining cell-free DNA topology further includes sequencing the second strand of the cell-free DNA duplex to generate a second sequence read; and determining, by one or more processors, one or more bases in the second sequence read to be soft-clipped. In some embodiments, determining the one or more bases in the second sequence read to be soft-clipped comprises aligning the second sequence to a reference sequence and identifying an unaligned portion of the second sequence read. In some embodiments, the first sequence read and the second sequence read are associated through a unique molecular identifier (UMI). In some embodiments, determining the one or more bases in the first sequence read to be soft-clipped comprises aligning the first sequence to the second sequence read and identifying an unaligned portion of the first sequence read. In some embodiments, determining the one or more bases in the second sequence read to be soft-clipped comprises aligning the second sequence to the first sequence read and identifying an unaligned portion of the second sequence read.

In some embodiments of the above methods, the method further includes extending a 3′ end of a second strand of the cell-free DNA duplex with inosine bases to fill a 5′ overhang of the first strand of the cell-free DNA duplex; attaching the second sequencing adapter to the cell-free DNA duplex; soft clipping, by the one or more processors, bases corresponding to a 3′ inosine extension of the second strand of the cell-free DNA duplex from the second sequence read; and determining, by the one or more processors, a length or sequence of the 5′ overhang of the first strand of the cell-free DNA duplex based on the soft clipping of the 3′ inosine extension of the second strand of the cell-free DNA duplex. In some embodiments, the method further includes detecting, by the one or more processors, a presence or absence of a disease based on the length or sequence of 5′ overhang of the first strand of the cell-free DNA duplex.

In some embodiments of the above method, extending the 3′ end of the first strand of the cell-free DNA duplex comprises forming a 3′ single inosine overhang. In some embodiments, the sequencing adapter comprises a 3′ cytosine overhang that complements to the 3′ inosine overhang.

In some embodiments, the method for determining cell-free DNA topology further includes amplifying the cell-free DNA duplex to attach a sample index to the cell-free DNA duplex.

In some embodiments of the above methods, the sequencing adapter or the second sequencing adapter comprises a sample index.

In some embodiments of the above methods, the sequencing adapter or the second sequencing adapter comprises the UMI.

In some embodiments of the above methods, the sequencing adapter or the second sequencing adapter is a Y-shaped sequencing adapter. In some embodiments, the adapter may be a Y full length adapter (e.g., a Y-shaped adapter comprising an index sequence), a stubby adapter, or a hairpin adapter.

Also provided herein is a method of making a sequencing construct, comprising: attaching a sequencing adapter to a 3′ overhang of a first strand of a cell-free DNA duplex; extending a 3′ end of the sequencing adapter with inosine bases to fill the 3′ overhang of the first strand of the cell-free DNA duplex; and attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex. In some embodiments, attaching the sequencing adapter to the 3′ overhang of the first strand of the cell-free DNA duplex comprises: extending the 3′ overhang of the first strand of the cell-free DNA duplex to provide a 3′ extension, wherein the sequencing adapter comprises a 3′ overhang that complements the 3′ extension of the first strand of the cell-free DNA duplex; and attaching the sequencing adapter to the 3′ extension of the first strand of the cell-free DNA duplex. In some embodiments, the 3′ overhang of the first strand of the cell-free DNA duplex is extended using nucleotide bases of the same base type.

Further described herein is a method for determining cell-free DNA topology, comprising: making the sequencing construct according to the above method; sequencing the second strand of the cell-free DNA duplex to generate a first sequence read; determining, by one or more processors, one or more bases in the first sequence read to be soft-clipped; soft clipping, by the one or more processors, bases corresponding to the 5′ inosine extension of the second strand of the cell-free DNA duplex from the first sequence read; and determining, by the one or more processors, a length or sequence of the 3′ overhang of the first strand of the cell-free DNA duplex based on the soft clipping of the 5′ inosine extension of the second strand of cell-free DNA duplex. In some embodiments, the method further comprises detecting, by the one or more processors, a presence or absence of disease (such as cancer) based on the length or sequence of the 3′ overhang of the first strand of the cell-free DNA duplex. In some embodiments, determining the one or more bases in the first sequence read to be soft-clipped comprises aligning the first sequence read to a reference sequence and identifying an unaligned portion of the first sequence read.

In some embodiments of the method for determining cell-free DNA topology, the method further comprises sequencing the first strand of the cell-free DNA duplex to generate a second sequence read; and determining, by one or more processors, one or more bases in the second sequence read to be soft-clipped. In some embodiments, determining the one or more bases in the second sequence read to be soft-clipped comprises aligning the second sequence to a reference sequence and identifying an unaligned portion of the second sequence read. In some embodiments, the first sequence read and the second sequence read are associated through a unique molecular identifier (UMI). In some embodiments, determining the one or more bases in the first sequence read to be soft-clipped comprises aligning the first sequence to the second sequence read and identifying an unaligned portion of the first sequence read. In some embodiments, determining the one or more bases in the second sequence read to be soft-clipped comprises aligning the second sequence to the first sequence read and identifying an unaligned portion of the second sequence read.

In some embodiments of the method for determining cell-free DNA topology, the method further comprises attaching a second sequencing adapter to a 3′ overhang of the second strand of a cell-free DNA duplex; extending a 3′ end of the second sequencing adapter with inosine bases to fill the 3′ overhang of the second strand of the cell-free DNA duplex; and attaching a 3′ inosine-extended end of the second sequencing adapter to a 5′ end of a first strand of the cell-free DNA duplex, wherein the 3′ inosine-extended end provides a 5′ inosine extension of the first strand of the cell-free DNA duplex. In some embodiments, the method further comprises sequencing the first strand of the cell-free DNA duplex to generate a second sequence read; determining, by one or more processors, one or more bases attached to the 5′ end of the second sequence read to be soft-clipped; soft clipping, by the one or more processors, bases corresponding to a 5′ inosine extension of the first strand of the cell-free DNA duplex from the second sequence read; and determining, by the one or more processors, a length or sequence of the 3′ overhang of the second strand of the cell-free DNA duplex based on the soft clipping of the 5′ inosine extension of the first strand of the cell-free DNA duplex. In some embodiments, the method further comprises detecting, by the one or more processors, a presence or absence of disease based on the length or sequence of the 3′ overhang of the second strand of the cell-free DNA duplex. In some embodiments, attaching the second sequencing adapter to the 3′ overhang of the second strand of a cell-free DNA duplex comprises attaching a single inosine to the 3′ overhang of the second strand of a cell-free DNA duplex and attaching the second sequencing adapter to the single inosine. In some embodiments, the sequencing adapter comprises a 3′ cytosine overhang that complements to the inosine attached to the 3′ overhang of the second strand of a cell-free DNA duplex. In some embodiments, determining the one or more bases attached to the 5′ end of the second sequence read to be soft-clipped comprises aligning the second sequence read to a reference sequence and identifying an unaligned portion of the second sequence read. In some embodiments, the first sequence read and the second sequence read are associated through a unique molecular identifier (UMI). In some embodiments, determining the one or more bases attached to the 5′ end of the second sequence read to be soft-clipped comprises aligning the second sequence read to the first sequence read and identifying an unaligned portion of the second sequence read. In some embodiments, determining the one or more bases attached to the 5′ end of the first sequence read to be soft-clipped comprises aligning the first sequence read to the second sequence read and identifying an unaligned portion of the first sequence read.

In some embodiments of the above method, amplifying the cell-free DNA duplex to attach a sample index to the cell-free DNA duplex.

In some embodiments of the above method, the sequencing adapter or the second sequencing adapter comprises a sample index.

In some embodiments of the above method, the sequencing adapter or the second sequencing adapter comprises the UMI.

In some embodiments of the above method, the sequencing adapter or the second sequencing adapter is a Y-shaped sequencing adapter. In some embodiments, the adapter may be a Y full length adapter (e.g., a Y-shaped adapter comprising an index sequence), a stubby adapter, or a hairpin adapter.

In some embodiments of the above method, the cell-free DNA duplex is obtained from a subject suspected of having cancer or determined to have cancer.

In some embodiments of any of the above methods, the method further comprises treating the subject with an anti-cancer therapy.

In some embodiments of any of the above methods, the method further comprises obtaining the cell-free DNA duplex from a subject.

In some embodiments of any of the above methods, the method further comprises the cell-free DNA duplex is obtained from a liquid biopsy sample. In some embodiments, the sample is a liquid biopsy sample and comprises blood, plasma, cerebrospinal fluid, sputum, stool, urine, or saliva. In some embodiments, the cell-free DNA duplex is a circulating tumor DNA (ctDNA) duplex.

In some embodiments of any of the above methods, the sequencing adapter or the second sequencing adapter comprise an amplification primer binding site, a flow cell adapter sequence, or a substrate adapter sequence.

In some embodiments of any of the above methods, the method further comprises amplifying the first strand and the second strand of the cell-free DNA duplex. In some embodiments, the amplifying comprises performing a polymerase chain reaction (PCR) amplification technique, a non-PCR amplification technique, or an isothermal amplification technique.

In some embodiments of any of the above methods, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencing is performed using a next generation sequencer.

In some embodiments of any of the above methods, the method further includes generating, by one or more processors, a report indicating the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex. In some embodiments, the method further includes transmitting the report to a healthcare provider. In some embodiments, the report is transmitted via a computer network or a peer-to-peer connection.

In some embodiments of any of the above methods, the method further includes generating a genomic profile for the subject comprising the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test.

Also described herein is a system comprising: one or more processors; and a memory communicatively coupled to the one or more processors and configured to store instructions that, when executed by the one or more processors, cause the system to: receive a first sequence read obtained by extending a 3′ end of a first strand of a cell-free DNA duplex with inosine bases to fill a 5′ overhang of a second strand of the cell-free DNA duplex; attaching a sequencing adapter to the cell-free DNA duplex; and sequencing the first strand of the cell-free DNA duplex to generate a first sequence read; determine one or more bases in the first sequence read to be soft-clipped; soft clip bases corresponding to a 3′ inosine extension of the first strand of the cell-free DNA duplex from the first sequence read; and determine a length or sequence of the 5′ overhang of the second strand of the cell-free DNA duplex based on the soft clipping of the 3′ inosine extension of the first strand of the cell-free DNA duplex. In some embodiments, the instructions further cause the system to detect a presence or absence of a disease (such as cancer) based on the length or sequence of the 5′ overhang of the second strand of the cell-free DNA duplex. In some embodiments, the first sequence read is further obtained by attaching a second sequencing adapter to a 3′ overhang of the second strand of the cell-free DNA duplex; extending a 3′ end of the second sequencing adapter with inosine bases to fill the 3′ overhang of the second strand of the cell-free DNA duplex; and attaching a 3′ inosine-extended end of the second sequencing adapter to a 5′ end of the first strand of cell-free DNA duplex, wherein the attached 3′ inosine-extended end provides a 5′ inosine extension of the first strand cell-free DNA duplex.

In some embodiments of the above system, the instructions, when executed by the one or more processors, further cause the system to: soft clip bases corresponding to the 5′ inosine extension of the first strand of the cell-free DNA duplex from the first sequence read; and determine a length or sequence of the 3′ overhang of the second strand of the cell-free DNA duplex based on the soft clipping of the 5′ inosine extension of the first strand of cell-free DNA duplex. In some embodiments, the instructions further cause the system to detect a presence or absence of a disease based on the length or sequence of the 3′ overhang of the second strand of the cell-free DNA duplex. In some embodiments, attaching the second sequencing adapter to the 3′ overhang of the second strand of the cell-free DNA duplex comprises: extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and attaching the second sequencing adapter to the 3′ extension of the second strand of the cell-free DNA duplex. In some embodiments, the 3′ overhang of the second strand of the cell-free DNA duplex is extended using nucleotide bases of the same base type.

In some embodiments of the above system, the one or more bases in the first sequence read is determined to be soft-clipped by a method comprising aligning the first sequence read to a reference sequence and identifying an unaligned portion of the first sequence read.

In some embodiments of the above system, the instructions, when executed by the one or more processors, further cause the system to: receive a second sequence read obtained by sequencing the second strand of the cell-free DNA duplex; and determine one or more bases in the second sequence read to be soft-clipped. In some embodiments, the one or more bases in the second sequence read are determined to be soft-clipped according to a method comprising aligning the second sequence to a reference sequence and identifying an unaligned portion of the second sequence read. In some embodiments, the first sequence read and the second sequence read are associated through a unique molecular identifier (UMI). In some embodiments, the one or more bases in the first sequence read are determined to be soft-clipped according to a method comprising aligning the first sequence to the second sequence read and identifying an unaligned portion of the first sequence read. In some embodiments, the one or more bases in the second sequence read are determined to be soft-clipped according to a method comprising aligning the second sequence to the first sequence read and identifying an unaligned portion of the second sequence read. In some embodiments, the second sequence read is further obtained by extending a 3′ end of a second strand of the cell-free DNA duplex with inosine bases to fill a 5′ overhang of the first strand of the cell-free DNA duplex; and attaching the second sequencing adapter to the cell-free DNA duplex. In some embodiments, the instructions, when executed by the one or more processors, further cause the system to: soft clip bases corresponding to a 3′ inosine extension of the second strand of the cell-free DNA duplex from the second sequence read; and determine a length or sequence of the 5′ overhang of the first strand of the cell-free DNA duplex based on the soft clipping of the 3′ inosine extension of the second strand of the cell-free DNA duplex.

In some embodiments of the above system, extending the 3′ end of the first strand of the cell-free DNA duplex comprises forming a 3′ single inosine overhang. In some embodiments, the sequencing adapter comprises a 3′ cytosine overhang that complements to the 3′ single inosine overhang.

Further described herein is a system comprising: one or more processors; and a memory communicatively coupled to the one or more processors and configured to store instructions that, when executed by the one or more processors, cause the system to: receive a first sequence read obtained by attaching a sequencing adapter to a 3′ overhang of a first strand of a cell-free DNA duplex; extending a 3′ end of the sequencing adapter with inosine bases to fill the 3′ overhang of the first strand of the cell-free DNA duplex; and attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex; determine one or more bases in the first sequence read to be soft-clipped; soft clip bases corresponding to the 5′ inosine extension of the second strand of the cell-free DNA duplex from the first sequence read; and determine a length or sequence of the 3′ overhang of the first strand of the cell-free DNA duplex based on the soft clipping of the 5′ inosine extension of the second strand of cell-free DNA duplex. In some embodiments, the instructions further cause the system to detect a presence or absence of a disease (such as cancer) based on the length or sequence of the 3′ overhang of the first strand of the cell-free DNA duplex.

In some embodiments of the above system, attaching the second sequencing adapter to the 3′ overhang of the second strand of the cell-free DNA duplex comprises: extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and attaching the second sequencing adapter to the 3′ extension of the second strand of the cell-free DNA duplex. In some embodiments, the 3′ overhang of the second strand of the cell-free DNA duplex is extended using nucleotide bases of the same base type.

In some embodiments of the above system, the one or more bases in the first sequence read is determined to be soft-clipped by a method comprising aligning the first sequence read to a reference sequence and identifying an unaligned portion of the first sequence read.

In some embodiments of the above system, the instructions, when executed by the one or more processors, further cause the system to: receive a second sequence read obtained by sequencing the second strand of the cell-free DNA duplex, and determine one or more bases in the second sequence read to be soft-clipped. In some embodiments, the one or more bases in the second sequence read are determined to be soft-clipped according to a method comprising aligning the second sequence to a reference sequence and identifying an unaligned portion of the second sequence read. In some embodiments, the first sequence read and the second sequence read are associated through a unique molecular identifier (UMI). In some embodiments, the one or more bases in the first sequence read are determined to be soft-clipped according to a method comprising aligning the first sequence to the second sequence read and identifying an unaligned portion of the first sequence read. In some embodiments, the one or more bases in the second sequence read are determined to be soft-clipped according to a method comprising aligning the second sequence to the first sequence read and identifying an unaligned portion of the second sequence read. In some embodiments, the second sequence read is further obtained by: attaching a second sequencing adapter to a 3′ overhang of the second strand of a cell-free DNA duplex; extending a 3′ end of the second sequencing adapter with inosine bases to fill the 3′ overhang of the second strand of the cell-free DNA duplex; and attaching a 3′ inosine-extended end of the second sequencing adapter to a 5′ end of a first strand of the cell-free DNA duplex, wherein the 3′ inosine-extended end provides a 5′ inosine extension of the first strand of the cell-free DNA duplex. In some embodiments, the instructions, when executed by the one or more processors, further cause the system to: soft clip bases corresponding to a 3′ inosine extension of the first strand of the cell-free DNA duplex from the second sequence read; and determine a length or sequence of the 5′ overhang of the second strand of the cell-free DNA duplex based on the soft clipping of the 3′ inosine extension of the first strand of the cell-free DNA duplex. In some embodiments, attaching the second sequencing adapter to the 3′ overhang of the second strand of a cell-free DNA duplex comprises attaching a single inosine to the 3′ overhang of the second strand of a cell-free DNA duplex and attaching the second sequencing adapter to the single inosine. In some embodiments, the sequencing adapter comprises a 3′ cytosine overhang that complements to the single inosine attached to the 3′ overhang of the second strand of a cell-free DNA duplex.

In some embodiments of any of the above systems, the sequencing adapter or the second sequencing adapter comprises the UMI.

In some embodiments of any of the above systems, the sequencing adapter or the second sequencing adapter is a Y-shaped sequencing adapter. In some embodiments, the adapter may be a Y full length adapter (e.g., a Y-shaped adapter comprising an index sequence), a stubby adapter, or a hairpin adapter.

In some embodiments of any of the above systems, the system further includes a nucleic acid amplifier configured to amplify the cell-free DNA duplex to attach a sample index to the cell-free DNA duplex. In some embodiments, the nucleic acid amplifier is a thermal cycler.

In some embodiments of any of the above systems, the cell-free DNA duplex is obtained from a subject suspected of having cancer or determined to have cancer.

In some embodiments of any of the above systems, the cell-free DNA duplex is obtained from a subject.

In some embodiments of any of the above systems, the cell-free DNA duplex is obtained from a liquid biopsy sample.

In some embodiments of any of the above systems, the sample is a liquid biopsy sample and comprises blood, plasma, cerebrospinal fluid, sputum, stool, urine, or saliva.

In some embodiments of any of the above systems, the cell-free DNA duplex is a circulating tumor DNA (ctDNA) duplex.

In some embodiments of any of the above systems, the sequencing adapter or the second sequencing adapter comprise an amplification primer binding site, a flow cell adapter sequence, or a substrate adapter sequence.

In some embodiments of any of the above systems, the system further includes a nucleic acid amplifier configured to amplify the first strand and the second strand of the cell-free DNA duplex. In some embodiments, the amplifying comprises performing a polymerase chain reaction (PCR) amplification technique, a non-PCR amplification technique, or an isothermal amplification technique.

In some embodiments of any of the above systems, the system further includes a sequencer configured to sequence the first strand of the cell-free DNA duplex and/or the second strand of the cell-free DNA duplex. In some embodiments, the sequencer is configured for massively parallel sequencing (MPS) technique, whole genome sequencing (WGS), whole exome sequencing, targeted sequencing, direct sequencing, or Sanger sequencing technique. In some embodiments, the sequencer is configured for massively parallel sequencing, and the massively parallel sequencing technique comprises next generation sequencing (NGS). In some embodiments, the sequencer is a next generation sequencer.

In some embodiments of any of the above systems, the instructions, when executed by the one or more processors, further cause the system to generate a report indicating the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex. In some embodiments, the instructions, when executed by the one or more processors, further cause the system to transmit the report to a healthcare provider. In some embodiments, the report is transmitted via a computer network or a peer-to-peer connection.

In some embodiments of any of the above systems, the instructions, when executed by the one or more processors, further cause the system to generate, by the one or more processors, a genomic profile for the subject comprising the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test.

Also described herein is a non-transitory computer-readable storage medium storing one or more programs, the one or more programs comprising instructions, which when executed by one or more processors of a system, cause the system to: receive a first sequence read obtained by extending a 3′ end of a first strand of a cell-free DNA duplex with inosine bases to fill a 5′ overhang of a second strand of the cell-free DNA duplex; attaching a sequencing adapter to the cell-free DNA duplex; and sequencing the first strand of the cell-free DNA duplex to generate a first sequence read; determine one or more bases in the first sequence read to be soft-clipped; soft clip bases corresponding to a 3′ inosine extension of the first strand of the cell-free DNA duplex from the first sequence read; and determine a length or sequence of the 5′ overhang of the second strand of the cell-free DNA duplex based on the soft clipping of the 3′ inosine extension of the first strand of the cell-free DNA duplex. In some embodiments, the instructions further cause the system to detect a presence or absence of a disease (such as cancer) based on the length or sequence of the 5′ overhang of the second strand of the cell-free DNA duplex.

In some embodiments of the above storage medium, wherein the first sequence read is further obtained by attaching a second sequencing adapter to a 3′ overhang of the second strand of the cell-free DNA duplex; extending a 3′ end of the second sequencing adapter with inosine bases to fill the 3′ overhang of the second strand of the cell-free DNA duplex; and attaching a 3′ inosine-extended end of the second sequencing adapter to a 5′ end of the first strand of cell-free DNA duplex, wherein the attached 3′ inosine-extended end provides a 5′ inosine extension of the first strand cell-free DNA duplex. In some embodiments, the instructions, when executed by the one or more processors, further cause the system to: soft clip bases corresponding to the 5′ inosine extension of the first strand of the cell-free DNA duplex from the first sequence read; and determine a length or sequence of the 3′ overhang of the second strand of the cell-free DNA duplex based on the soft clipping of the 5′ inosine extension of the first strand of cell-free DNA duplex. In some embodiments, the instructions further cause the system to detect a presence or absence of a disease based on the length or sequence of the 3′ overhang of the second strand of the cell-free DNA duplex.

In some embodiments of the above storage medium, attaching the second sequencing adapter to the 3′ overhang of the second strand of the cell-free DNA duplex comprises: extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and attaching the second sequencing adapter to the 3′ extension of the second strand of the cell-free DNA duplex. In some embodiments, the 3′ overhang of the second strand of the cell-free DNA duplex is extended using nucleotide bases of the same base type.

In some embodiments of the above storage medium, the one or more bases in the first sequence read is determined to be soft-clipped by a method comprising aligning the first sequence read to a reference sequence and identifying an unaligned portion of the first sequence read.

In some embodiments of the above storage medium, the instructions, when executed by the one or more processors, further cause the system to: receive a second sequence read obtained by sequencing the second strand of the cell-free DNA duplex, and determine one or more bases in the second sequence read to be soft-clipped. In some embodiments, the one or more bases in the second sequence read are determined to be soft-clipped according to a method comprising aligning the second sequence to a reference sequence and identifying an unaligned portion of the second sequence read. In some embodiments, the first sequence read and the second sequence read are associated through a unique molecular identifier (UMI). In some embodiments, the one or more bases in the first sequence read are determined to be soft-clipped according to a method comprising aligning the first sequence to the second sequence read and identifying an unaligned portion of the first sequence read. In some embodiments, the one or more bases in the second sequence read are determined to be soft-clipped according to a method comprising aligning the second sequence to the first sequence read and identifying an unaligned portion of the second sequence read. In some embodiments, the second sequence read is further obtained by extending a 3′ end of a second strand of the cell-free DNA duplex with inosine bases to fill a 5′ overhang of the first strand of the cell-free DNA duplex; and attaching the second sequencing adapter to the cell-free DNA duplex. In some embodiments, the instructions, when executed by the one or more processors, further cause the system to: soft clip bases corresponding to a 3′ inosine extension of the second strand of the cell-free DNA duplex from the second sequence read; and determine a length or sequence of the 5′ overhang of the first strand of the cell-free DNA duplex based on the soft clipping of the 3′ inosine extension of the second strand of the cell-free DNA duplex.

In some embodiments of the above storage medium, extending the 3′ end of the first strand of the cell-free DNA duplex comprises forming a 3′ single inosine overhang. In some embodiments, the sequencing adapter comprises a 3′ cytosine overhang that complements to the 3′ single inosine overhang.

Further described herein is a non-transitory computer-readable storage medium storing one or more programs, the one or more programs comprising instructions, which when executed by one or more processors of a system, cause the system to: receive a first sequence read obtained by attaching a sequencing adapter to a 3′ overhang of a first strand of a cell-free DNA duplex; extending a 3′ end of the sequencing adapter with inosine bases to fill the 3′ overhang of the first strand of the cell-free DNA duplex; and attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex; determine one or more bases in the first sequence read to be soft-clipped; soft clip bases corresponding to the 5′ inosine extension of the second strand of the cell-free DNA duplex from the first sequence read; and determine a length or sequence of the 3′ overhang of the first strand of the cell-free DNA duplex based on the soft clipping of the 5′ inosine extension of the second strand of cell-free DNA duplex. In some embodiments, the instructions further cause the system to detect a presence or absence of a disease (such as cancer) based on the length or sequence of the 3′ overhang of the first strand of the cell-free DNA duplex.

In some embodiments of the above storage medium, attaching the second sequencing adapter to the 3′ overhang of the second strand of the cell-free DNA duplex comprises: extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and attaching the second sequencing adapter to the 3′ extension of the second strand of the cell-free DNA duplex. In some embodiments, the 3′ overhang of the second strand of the cell-free DNA duplex is extended using nucleotide bases of the same base type.

In some embodiments of the above storage medium, the one or more bases in the first sequence read is determined to be soft-clipped by a method comprising aligning the first sequence read to a reference sequence and identifying an unaligned portion of the first sequence read.

In some embodiments of the above storage medium, the instructions, when executed by the one or more processors, further cause the system to: receive a second sequence read obtained by sequencing the second strand of the cell-free DNA duplex, and determine one or more bases in the second sequence read to be soft-clipped. In some embodiments, the one or more bases in the second sequence read are determined to be soft-clipped according to a method comprising aligning the second sequence to a reference sequence and identifying an unaligned portion of the second sequence read. In some embodiments, the first sequence read and the second sequence read are associated through a unique molecular identifier (UMI). In some embodiments, the one or more bases in the first sequence read are determined to be soft-clipped according to a method comprising aligning the first sequence to the second sequence read and identifying an unaligned portion of the first sequence read. In some embodiments, the one or more bases in the second sequence read are determined to be soft-clipped according to a method comprising aligning the second sequence to the first sequence read and identifying an unaligned portion of the second sequence read. In some embodiments, the second sequence read is further obtained by: attaching a second sequencing adapter to a 3′ overhang of the second strand of a cell-free DNA duplex; extending a 3′ end of the second sequencing adapter with inosine bases to fill the 3′ overhang of the second strand of the cell-free DNA duplex; attaching a 3′ inosine-extended end of the second sequencing adapter to a 5′ end of a first strand of the cell-free DNA duplex, wherein the 3′ inosine-extended end provides a 5′ inosine extension of the first strand of the cell-free DNA duplex. In some embodiments, the instructions, when executed by the one or more processors, further cause the system to: soft clip bases corresponding to a 3′ inosine extension of the first strand of the cell-free DNA duplex from the second sequence read; and determine a length or sequence of the 5′ overhang of the second strand of the cell-free DNA duplex based on the soft clipping of the 3′ inosine extension of the first strand of the cell-free DNA duplex. In some embodiments, attaching the second sequencing adapter to the 3′ overhang of the second strand of a cell-free DNA duplex comprises attaching a single inosine to the 3′ overhang of the second strand of a cell-free DNA duplex and attaching the second sequencing adapter to the inosine. In some embodiments, the sequencing adapter comprises a 3′ cytosine overhang that complements to the single inosine attached to the 3′ overhang of the second strand of a cell-free DNA duplex.

In some embodiments of the above storage medium, the sequencing adapter or the second sequencing adapter comprises the UMI.

In some embodiments of the above storage medium, the sequencing adapter or the second sequencing adapter is a Y-shaped sequencing adapter. In some embodiments, the adapter may be a Y full length adapter (e.g., a Y-shaped adapter comprising an index sequence), a stubby adapter, or a hairpin adapter.

In some embodiments of the above storage medium, the cell-free DNA duplex is obtained from a subject suspected of having cancer or determined to have cancer.

In some embodiments of the above storage medium, the cell-free DNA duplex is obtained from a subject.

In some embodiments of the above storage medium, the cell-free DNA duplex is obtained from a liquid biopsy sample. For example, in some embodiments, the sample is a liquid biopsy sample and comprises blood, plasma, cerebrospinal fluid, sputum, stool, urine, or saliva.

In some embodiments of the above storage medium, the cell-free DNA duplex is a circulating tumor DNA (ctDNA) duplex.

In some embodiments of the above storage medium, the sequencing adapter or the second sequencing adapter comprise an amplification primer binding site, a flow cell adapter sequence, or a substrate adapter sequence.

In some embodiments of the above storage medium, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencing is performed using a next generation sequencer.

In some embodiments of the above storage medium, the instructions, when executed by the one or more processors, further cause the system to generate a report indicating the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex. In some embodiments, the instructions, when executed by the one or more processors, further cause the system to transmit the report to a healthcare provider. In some embodiments, the report is transmitted via a computer network or a peer-to-peer connection.

In some embodiments of the above storage medium, the instructions, when executed by the one or more processors, further cause the system to generate, by the one or more processors, a genomic profile for the subject comprising the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A shows an exemplary process for making a sequencing construct and determining a 5′ overhang length from a DNA duplex molecule (e.g., a cfDNA molecule), according to some embodiments.

FIG. 1B shows an exemplary embodiment of the process for making a sequencing construct and determining a 5′ overhang length from a DNA duplex molecule (e.g., a cfDNA molecule) shown in FIG. 1A. In the exemplary method, 5′ overhangs in cfDNA are filled in by deoxyinosine before adapters are attached to the ends of the cfDNA. Optionally, the cfDNA is PCR amplified before sequencing. The sequences are then aligned and analyzed for overhang length and sequence.

FIG. 2A shows an exemplary method for making a sequencing construct useful for determining a 3′ overhang length from a DNA duplex, such as a cfDNA molecule, according to some embodiments.

FIG. 2B shows an exemplary method for determining a 3′ overhang length from a DNA duplex, such as a cfDNA molecule, according to some embodiments.

FIG. 3A shows an exemplary method for making a sequencing construct useful for determining a 5′ and/or a 3′ overhang length from a DNA duplex molecule (such as a cfDNA molecule), along with a process for determining the 5′ and/or a 3′ overhang length for the DNA duplex molecule, according to some embodiments.

FIG. 3B shows an exemplary embodiment of the method for making a sequencing construct and determining a 5′ and/or a 3′ overhang length from a DNA duplex molecule (such as a cfDNA molecule), as shown in FIG. 3A, according to some embodiments. In the exemplary method, 5′ overhangs in cfDNA are filled in by deoxyinosine. One adapter is attached to a 3′ overhang of the bottom strand, and another adapter is attached to the opposite end of the molecule. The cfDNA 3′ overhangs is then filled in by deoxyinosine. Optionally, the cfDNA is PCR amplified before sequencing. The sequences are then aligned and analyzed for overhang length and sequence.

FIG. 3C shows another exemplary embodiment of the method for making a sequencing contract and determining a 5′ and/or a 3′ overhang length from a DNA duplex molecule (such as a cfDNA molecule), as shown in FIG. 3A, according to some embodiments. The aspects of the method shown in FIG. 3C may be readily applied to other embodiments of the methods described herein, including the process shown in FIGS. 1A, 1B, 2A, and 2B. In the exemplary method, 5′ overhangs in cfDNA are filled in by deoxyinosine and then the first adapter is attached to the cfDNA at the deoxyinosine-filled end. The 3′ overhang ends in cfDNA are extended with a single type of nucleotide, represented by “Y,” and the second adapter is attached to the cfDNA and the cfDNA 3′ overhangs are filled in by deoxyinosine. Optionally, the cfDNA is PCR amplified before sequencing. The sequences are then aligned and analyzed for overhang length and sequence.

FIG. 4 shows process that is used to determine cfDNA topology, according to some embodiments.

FIG. 5 shows an exemplary read processing matrix for extracting data from paired-end sequencing, according to some embodiments.

FIG. 6 depicts an exemplary computing device or system in accordance with one embodiment of the present disclosure.

FIG. 7 depicts an exemplary computer system or computer network, in accordance with some instances of the systems described herein.

### DETAILED DESCRIPTION

Described herein are methods and systems for determining a DNA duplex (e.g., cell-free DNA) molecule topology, in particular, the length and sequences of 5′ and/or 3′ overhangs. Standard methods of assessing cfDNA do not adequately capture cfDNA 5′ and 3′ overhang information to provide complete description of the topological information or characteristics of the cfDNA molecules, which may provide information related to the onset, progression, diagnosis, treatment, etc. of various diseases, in particular for cancers. The loss of this information is an obstacle not only to the understanding of diseases processes but also to the utilization of this information for the improvement of human health (e.g., early detection of diseases such as cancer, any association to treatment efficacies, etc.).

The methods and systems described herein provide for the generation and analysis of sequencing libraries that capture 5′ and/or 3′ overhangs including a length and/or sequence of the native ends of the DNA duplex molecule. In particular, the methods and systems described herein may further generate and analyze sequence libraries from the cfDNA collected from early-stage cancer patients or patients suspected of having cancer. The described methods and systems may therefore provide a solution to the limitations of other methods of analyzing cfDNA to better capture and utilize the information present in cfDNA molecules.

Accordingly, in one aspect, a method for determining cell-free DNA topology includes: extending a 3′ end of a first strand of a cell-free DNA duplex with inosine bases to fill a 5′ overhang of a second strand of the cell-free DNA duplex; attaching a sequencing adapter to the cell-free DNA duplex; sequencing the first strand of the cell-free DNA duplex to generate a first sequence read; determining, by one or more processors, one or more bases in the first sequence read to be soft-clipped; soft clipping, by the one or more processors, bases corresponding to a 3′ inosine extension of the first strand of the cell-free DNA duplex from the first sequence read; and determining, by the one or more processors, a length or sequence of the 5′ overhang of the second strand of the cell-free DNA duplex based on the soft clipping of the 3′ inosine extension of the first strand of the cell-free DNA duplex. The method may further include attaching a second sequencing adapter to a 3′ overhang of the second strand of the cell-free DNA duplex; extending a 3′ end of the second sequencing adapter with inosine bases to fill the 3′ overhang of the second strand of the cell-free DNA duplex; attaching a 3′ inosine-extended end of the second sequencing adapter to a 5′ end of the first strand of cell free DNA duplex, wherein the attached 3′ inosine-extended end provides a 5′ inosine extension of the first strand cell-free DNA duplex; determining, by one or more processors, one or more bases attached to the 5′ end of the first sequence read to be soft-clipped; soft clipping, by the one or more processors, bases corresponding to the 5′ inosine extension of the first strand of the cell-free DNA duplex from the first sequence read; and determining, by the one or more processors, a length or sequence of the 3′ overhang of the second strand of the cell-free DNA duplex based on the soft clipping of the 5′ inosine extension of the first strand of cell-free DNA duplex. In some embodiments, the method further includes detecting, by the one or more processors, a presence or absence of a disease (such as cancer) based on the length or sequence of the 5′ overhang of the first strand and/or second strand of the cell-free DNA duplex. In some embodiments, the method further comprises detecting, by the one or more processors, a presence or absence of a disease (such as cancer) based on the length or sequence of 3′ overhang of the first strand and/or second strand of the cell-free DNA duplex.

In another aspect, there is a method of making a sequencing construct, which includes attaching a sequencing adapter to a 3′ overhang of a first strand of a cell-free DNA duplex; extending a 3′ end of the sequencing adapter with inosine bases to fill the 3′ overhang of the first strand of the cell-free DNA duplex; and attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex. The method may further include analyzing the sequencing construct to determine a topology of the cell-free DNA duplex, for example by sequencing the second strand of the cell-free DNA duplex to generate a first sequence read; determining, by one or more processors, one or more bases in the first sequence read to be soft-clipped; soft clipping, by the one or more processors, bases corresponding to the 5′ inosine extension of the second strand of the cell-free DNA duplex from the first sequence read; and determining, by the one or more processors, a length or sequence of the 3′ overhang of the first strand of the cell-free DNA duplex based on the soft clipping of the 5′ inosine extension of the second strand of cell-free DNA duplex. In some embodiments, the method further includes detecting, by the one or more processors, a presence or absence of a disease (such as cancer) based on the length or sequence of the 5′ overhang of the first strand and/or second strand of the cell-free DNA duplex. In some embodiments, the method further comprises detecting, by the one or more processors, a presence or absence of a disease (such as cancer) based on the length or sequence of 3′ overhang of the first strand and/or second strand of the cell-free DNA duplex.

Also described are systems and computer readable storage media for performing the methods described herein.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated. Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

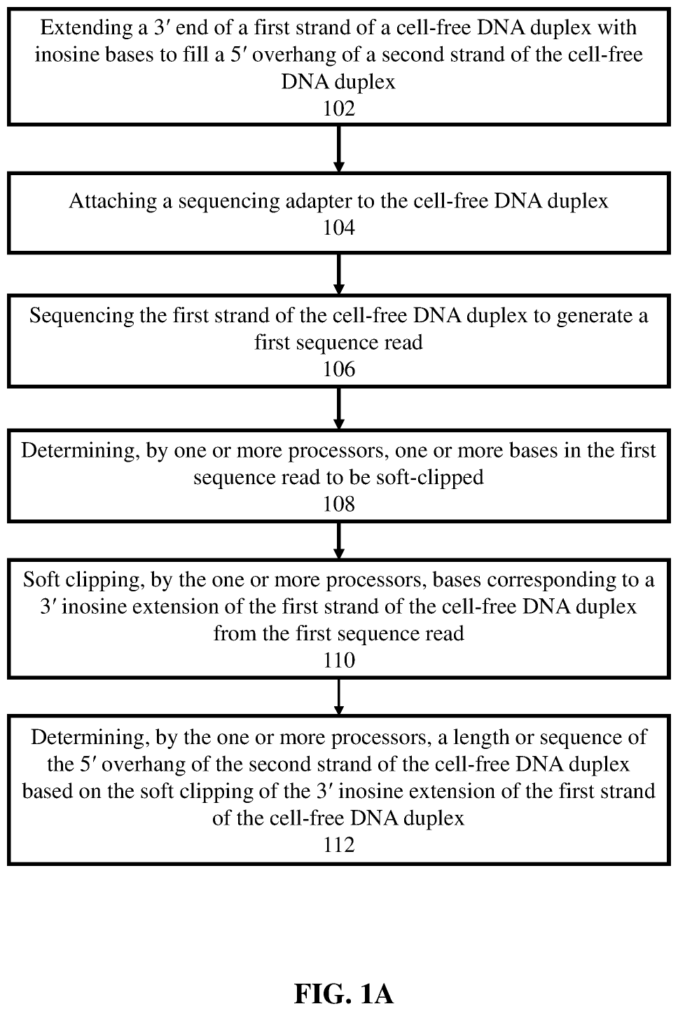

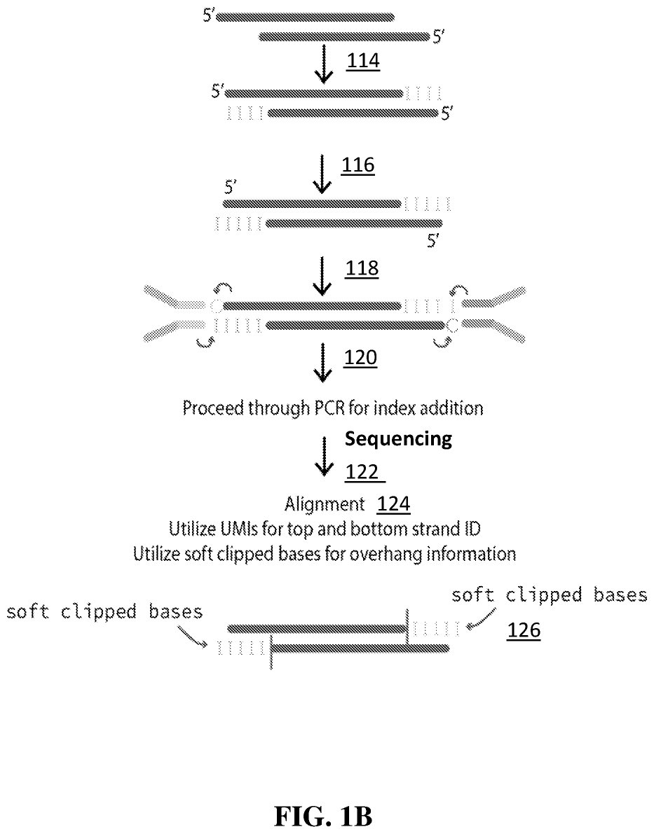

FIGS. 1-7 illustrate processes and systems according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Methods for Determining Overhang Topology

Provided herein is a method of generating a sequencing construct for a DNA duplex (e.g., a cfDNA molecule) and analyzing said sequencing construct to determine a topology for the DNA duplex molecule. For example, the ends of cfDNA may include 5′ and/or 3′ overhangs, and the length and/or sequence of such overhangs may be determined according to the methods described herein. For example, the methods may include filling in the overhang gaps using a nucleotide or analog thereof, such as by using an inosine nucleotide (e.g., deoxyinosine). Adapters can then be attached to ends of the cfDNA duplex molecule and the first and/or second strand can be sequenced, processed (e.g., the inosine bases soft-clipped), and analyzed for cfDNA topology, including 5′ and/or 3′ overhang length and sequence.

As further described herein, the method may include generation of a sequencing construct for the DNA duplex molecule (e.g., cfDNA). The sequencing construct may be made so that a 5′ overhang may be analyzed (e.g., length and/or sequence determined), a 3′ overhang may be analyzed (e.g., length and/or sequence determined), or both a 5′ and a 3′ overhang may be analyzed. Certain embodiments are described in reference to a single DNA duplex molecule (e.g., a single cfDNA duplex), but it is understood that the methods may be applied for the construction of a sequencing library where a plurality of DNA duplex molecules, which may have different topologies, may be made and/or analyzed in parallel.

In some instances, one or both strands of the cfDNA duplex may include 5′ and/or 3′ overhangs. The sequencing construct generation and analysis methods utilize a nucleic acid analog, for example deoxyinosine (dI), to fill in the 5′ and/or 3′ overhang gaps, thereby retaining overhang sequence and length data for further downstream applications. Deoxyinosine can be incorporated or added by a polymerase during extension, including but not limited to any standard or high fidelity formulation of Taq polymerases and variants thereof (e.g., LongAmp Taq, Epimark® Taq, Hemo Klen Taq, OneTaq), Bst DNA polymerases, Bsu DNA polymerases, phi29 DNA polymerases, T7 DNA polymerases, etc. Other polymerases may be used, such as Pfu, KOD, or Tth DNA polymerase.

Additional enzymes may be used to incorporate sequencing adapters onto each end of the cfDNA duplex. For example, in some embodiments, ligases may be used to attach the sequencing adapters to the cfDNA ends by ligation. In some embodiments, a terminal transferase, for example, a terminal deoxynucleotidyl transferase, may be used to extend the end of a 3′ overhang of a cfDNA molecule in order to generate a nucleotide tail with a known sequence to pair with an adapter. In some embodiments, the adapter comprises a sequence that is complementary to the extension sequence generated by a terminal transferase at the 3′ overhang end of a cfDNA molecule. The terminal transferase may attach one or more (e.g., a plurality of nucleotide bases to the 3′ overhang end of the cfDNA duplex molecule. The attached bases may be, for example, canonical bases (e.g., A, C, T, or G). In some embodiments, the attached bases are of the same nucleotide base type. The adapters may include sequencing primer binding sites and in some embodiments may further include a sample index sequence and/or UMI sequence. Once the adapters have been attached to the 5′ and 3′ ends of the cfDNA duplex, the first strand and/or second strand of the cfDNA duplex can then be sequenced to generate a first and/or a second sequence read.

Once a sequencing construct or sequencing library is made, the one or both strands of the DNA duplex molecule may be sequenced to generate one or more sequence reads. Sequencing may occur through any suitable process. In some implementations of the method, sequencing is performed by next-generation sequencing. “Next-generation sequencing” (or “NGS”) as used herein may also be referred to as “massively parallel sequencing” (or “MPS”) and refers to any sequencing method that determines the nucleotide sequence of either individual nucleic acid molecules (e.g., as in single molecule sequencing) or clonally expanded proxies for individual nucleic acid molecules in a high throughput fashion. Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some embodiments, the sequencing may comprise, for example, whole genome sequencing (WGS), whole exome sequencing, targeted sequencing or direct sequencing. In some embodiments, sequencing may be performed using, e.g., Sanger sequencing. In some instances, the sequencing may comprise a paired-end sequencing technique that allows both ends of a fragment to be sequenced and generates high-quality, mappable sequence data for detection of, e.g., cfDNA 5′ and/or 3′ overhang sequences.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some embodiments, sequencing may comprise Illumina MiSeq sequencing. In some embodiments, sequencing may comprise Illumina HiSeq sequencing. In some embodiments, sequencing may comprise Illumina NovaSeq sequencing. Optimized methods for sequencing nucleic acids extracted from a sample are described in more detail in, e.g., International Patent Application Publication No. WO 2020/236941, the entire content of which is incorporated herein by reference.

The inclusion of dI to fill in the overhang gaps provides a clear demarcation in each sequence read wherein the cfDNA sequence ends and the dI-filled sequence of the overhang gap begins. During amplification (e.g., PCR amplification), the dIs are converted to one or more nucleotides (e.g., A, T, G, C). During alignment, the nucleotides representing the jagged end (dIs fill in) will not align (map) to the reference genome sequence and, therefore, will identify where the original cfDNA sequence (e.g., fragment) ends and where the dIs filled sequence of the overhang gap begins. dI inclusion further leads to erroneous nucleotide pairing that can be determined by sequence alignment. The computational removal of the dI sequences by soft clipping provides information about the length of each cfDNA molecule's 5′ and/or 3′ overhang sequences. When combined with sequence alignment, the sequence of the 5′ and/or 3′ overhangs can be ascertained, as described in further detail below.

Sequencing reads may be analyzed to determine one or more bases in the sequence reads to be soft clipped. As further described herein, the methods may include the incorporation of inosine bases during the making of the sequencing construct. The soft clipping may be based on the presence of an inosine extension, which indicates an overhang on the complementary strand. Thus, the length of an overhang in one strand may be based on the soft clipping of one or more inosine extensions in the complementary strand. Given the length of an overhang and the sequence proximal to the overhang, the sequence of the overhang itself may then be determined.

The determination of one or more bases in a sequence read to be soft clipped may include aligning the sequence read with a reference sequence or to a sequence of a complementary strand. Complementary strands may be determined using standard molecular barcode (i.e., unique molecular identifier (UMI)) techniques. These UMI techniques may be either exogenously unique identifiers or non-unique identifiers, e.g., the exogenous barcodes added to the molecule are not unique, but after alignment, other characteristics of the molecule determined from the read, e.g., coordinates, a portion of the endogenous sequence, etc., are combined with the non-unique barcode to create a UMI. Alignment is the process of matching a query sequence read with one or more additional sequence reads or reference sequence. Alignment may further include mapping of the sequence read, for example to a location, e.g., a genomic location or locus, within a reference sequence. In some embodiments, a sequence reads may be aligned to a known reference sequence (e.g., a wild-type sequence). In some embodiments, the reference sequences can be obtained from databases of the human genome (e.g., the HG19 human reference genome) or cancer mutations (e.g., COSMIC). Methods of sequence alignment for sequence reads are described in, e.g., Trapnell, C. and Salzberg, S. L. Nature Biotech., 2009, 27:455-457. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

In some embodiments, the methods and systems disclosed herein may integrate the use of multiple, individually tuned, alignment methods or algorithms to optimize base-calling performance in sequencing methods, particularly in methods that rely on massively parallel sequencing (MPS). In some embodiments, the disclosed methods and systems may comprise the use of one or more global alignment algorithms. In some embodiments, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1): 195-197), the Striped Smith-Waterman algorithm (see, e.g., Farrar (2007), “Striped Smith-Waterman Speeds Database Searches Six Times Over Other SIMD Implementations”, Bioinformatics 23(2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

The disclosed methods and systems may be implemented using soft-clipping analytics. The first sequence and/or the second sequence can be aligned to a reference sequence, or the first sequence can be aligned to the second sequence read or vice versa. The 5′ and/or 3′ overhang sequence(s) wherein the overhang gaps are inosine-filled will not align with the reference sequence, and therefore these inosine-filled gaps are computationally trimmed from the first and/or second read of the first and/or second sequence of the cfDNA molecule. The 5′ overhang length and/or 3′ overhang length can be calculated based on the number of misaligned inosine bases that are soft-clipped. This information can then be further processed to identify additional DNA topological features, including the 5′ and/or 3′ overhang sequence, and can be analyzed for disease associations, such as cancer.

FIG. 1A shows an exemplary method for determining cell-free DNA topology, such as for determining a 5′ overhang length of a cell-free DNA duplex, according to some embodiments. As shown at 102 in FIG. 1A, the method comprises extending a 3′ end of a first strand of a cell-free DNA duplex with inosine bases to fill a 5′ overhang of a second strand of the cell-free DNA duplex. In some embodiments, the inosine is a deoxyinosine (dI). At 104, the method comprises the attaching of a sequencing adapter to the cell-free DNA duplex. In some embodiments, attaching of a sequencing adapter comprises ligating of the sequencing adapter to the cell-free DNA duplex. The sequencing adapter may be, for example, a Y-shaped sequencing adapter. In some embodiments, the adapter may be a Y full length adapter (e.g., a Y-shaped adapter comprising an index sequence), a stubby adapter, or a hairpin adapter. In some embodiments, making the sequencing construct can include amplifying the first strand and the second strand of the cell-free DNA duplex, for example prior to sequencing. The amplifying process can allow for the attachment of a sample index to the cell-free DNA duplex. In some embodiments, however, a sample index is included in the sequencing adapter, making the incorporation of a sample index in a downstream amplification process unnecessary. In some embodiments, the amplifying comprises performing a polymerase chain reaction (PCR) amplification technique, a non-PCR amplification technique, or an isothermal amplification technique.

At 106, the method comprises sequencing the first strand of the cell-free DNA duplex to generate a first sequence read. In some embodiments, the sequencing comprises next-generation sequencing (“NGS”). In some embodiments, the sequencing comprises paired-end sequencing. In some embodiments, the sequencing adapter or the second sequencing adapter comprise an amplification primer binding site, a flow cell adapter sequence, or a substrate adapter sequence. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencing is performed using a next generation sequencer.

At 108 of FIG. 1A, the method further includes determining, by one or more processors, one or more bases in the first sequence read to be soft-clipped. Determining the one or more bases in the first sequence read to be soft-clipped may include, for example, aligning the first sequence read to a reference sequence and identifying an unaligned portion of the first sequence read. The unaligned portion of the first sequence read can be associated with inosine bases in the extended first strand. Because these inosine bases are artificially generated, they do not align with the reference sequence or complementary strand, and therefore indicate an overhang in the complementary strand. These unaligned bases in the 3′ portion of the sequence read can therefore be identified computationally for soft clipping.

In some embodiments, the method includes sequencing the second strand of the cell-free DNA duplex to generate a second sequence read. The one or more bases in the first sequence read to be soft clipped may be determined by aligning the first sequence (i.e., for the first strand) to the second sequence read (i.e., for the second strand) and identifying an unaligned portion of the first sequence read. Matching the first sequence read to the second sequence read may include the use of a UMI common between the first sequence read and the second sequence read. The unaligned 3′ portion of the sequence read may be identified for soft clipping.

At 110 of FIG. 1A, the method comprises soft clipping, by the one or more processors, bases corresponding to a 3′ inosine extension of the first strand of the cell-free DNA duplex from the first sequence read. At 112, the method comprises determining, by the one or more processors, a length or sequence of the 5′ overhang of the second strand of the cell-free DNA duplex based on the soft clipping of the 3′ inosine extension of the first strand of the cell-free DNA duplex.

FIG. 1B shows an exemplary embodiment of the process described in FIG. 1A. The double stranded DNA duplex in the example shown in FIG. 1B includes a 5′ overhang in each of the top strand and the bottom strand, although the method may be performed when there is a 5′ overhang in only one strand or in both strands. In the shown example at 114, the 3′ ends of the top and bottom strands are extended using inosine bases (e.g., deoxy inosine), which fills the 5′ overhang. Optionally, a polymerase capable of attaching a single nucleotide (for example, an inosine base) to the 3′ end (e.g., blunt end) of the DNA duplex molecule may be used to generate a single 3′ nucleotide overhang (e.g., a single 3′ inosine overhang), as shown at 116 of FIG. 1B. The polymerase may be, for example, a Taq polymerase. In another embodiment, a terminal transferase may be used to attach one or more (e.g., a plurality of nucleotide bases to the 3′ overhang end of the cfDNA duplex molecule. The attached bases may be, for example, canonical bases (e.g., A, C, T, or G). The 3′ overhang may increase attachment efficiency of the sequencing adapter, which may include a 3′ overhang that complements the 3′ overhang of the cfDNA molecule. In some embodiments, the attached bases are of the same nucleotide base type. Sequencing adapters may then be attached to the DNA duplex molecule at 118. Optionally, if a tail is included on the 3′ end(s), the adapter may include a 3′ overhang that complements the tail. For example, if a single inosine tail is included on the 3′ end(s), the adapter may include a 3′ overhang comprising a cytosine base, as shown in FIG. 1B. This exemplary embodiment further shows the optional step involving the amplification of the first strand of the cell-free DNA duplex, for example by polymerase chain reaction (PCR). In this example, the process of amplification adds a sample index to the first and second strands of the cell-free DNA duplex. In some embodiments, the sequencing adapters includes a unique molecular identifier (UMI), which can allow for pairing the first strand (top) and the second strand (bottom).

Once the sequencing construct is prepared, as shown in the top portion of FIG. 1B, the nucleic acid molecules may be sequenced at 122 to provide sequencing reads. Optionally, the sequencing construct may be amplified prior to sequencing, as shown at 120, for example by PCR, which may allow for the incorporation of an index sequence such as a sample index. In the shown exemplary embodiment, the sequencing reads are aligned at 124, for example to a reference sequence. In another example, top and bottom strands of the same DNA duplex molecule are aligned to each other. Alignment may include the use of a unique molecular identifier (which may be, for example, included in the sequencing adapter), which allows the top and bottom strands to be matched to each other. The portion of the sequence read that does not align with the reference sequence or complementary strand may be identified for soft clipping as shown at 126. That is, bases that do not align with the reference sequence or complementary strand may be identified as inosine bases that were used to artificially extend the 3′ ends. The soft clipped bases may be used to determine the length of the 5′ overhang in the complementary strand, as the inosine bases were used to fill the 5′ overhang.

In some embodiments, there is provided a method for determining cell-free DNA topology, comprising: extending a 3′ end of a first strand of a cell-free DNA duplex with inosine bases to fill a 5′ overhang of a second strand of the cell-free DNA duplex; attaching a sequencing adapter to the cell-free DNA duplex; sequencing the first strand of the cell-free DNA duplex to generate a first sequence read; determining, by one or more processors, one or more bases in the first sequence read to be soft-clipped; soft clipping, by the one or more processors, bases corresponding to a 3′ inosine extension of the first strand of the cell-free DNA duplex from the first sequence read; and determining, by the one or more processors, a length or sequence of the 5′ overhang of the second strand of the cell-free DNA duplex based on the soft clipping of the 3′ inosine extension of the first strand of the cell-free DNA duplex. In some embodiments, extending the 3′ end of the first strand of the cell-free DNA duplex comprises forming a single 3′ inosine overhang. In some embodiments, the sequencing adapter comprises a 3′ nucleotide overhang (e.g., a cytosine overhang) that complements to the single 3′ inosine overhang.

In some embodiments, the method further comprises detecting, by the one or more processors, a presence or absence of a disease based on the length or sequence of the 5′ overhang of the second strand of the cell-free DNA duplex. In some embodiments, the disease is a cancer.

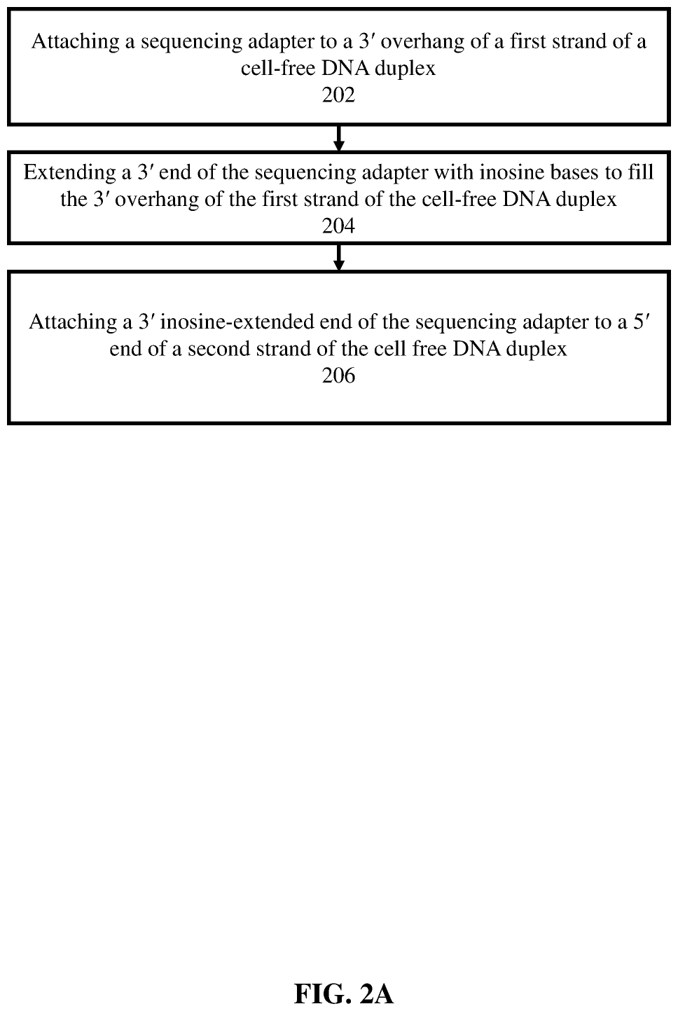

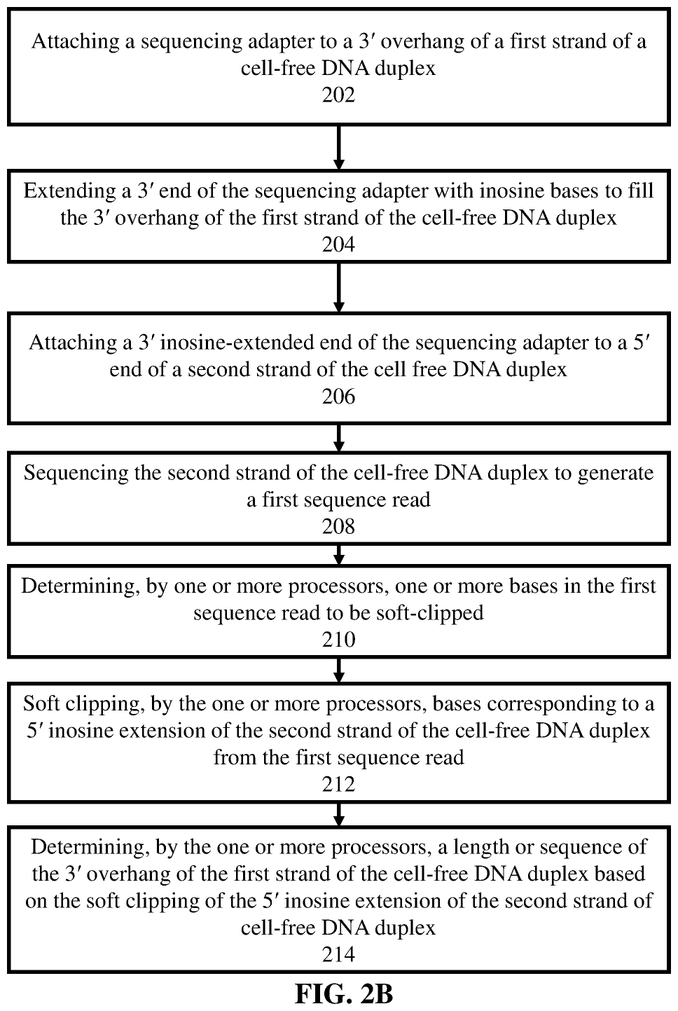

The methods described herein may additionally or alternatively include the making of a sequencing construct for analyzing a 3′ overhang of a DNA molecule, for example as shown in FIG. 2A. As shown at 202, the method includes attaching (for example, by ligating) a sequencing adapter to a 3′ overhang of a first strand of a cell-free DNA duplex. In some implementations, the sequencing adapter is ligated directly to the 3′ overhang of the first strand. In other implementations, the 3′ overhang of the first strand may be extended by one or more bases (i.e., to provide a 3′ extension or tail). The sequencing adapter may include a 3′ overhang that complements the 3′ extension of the first strand of the cell-free DNA duplex. Including an overhang on the adapter that complements the extension or tail attached to the 3′ overhang increases the efficiency of the ligation reaction. The 3′ extension or tail may be, for example, a single inosine. Inosine is an analogue of guanine and, accordingly, preferentially pairs with cytosine bases (although it may pair with other canonical nucleotide bases (i.e., adenine, thymine, or guanine)). If a single inosine tail is included on the 3′ end(s), the adapter may include, for example, a 3′ overhang comprising a cytosine base, which complements the inosine base.

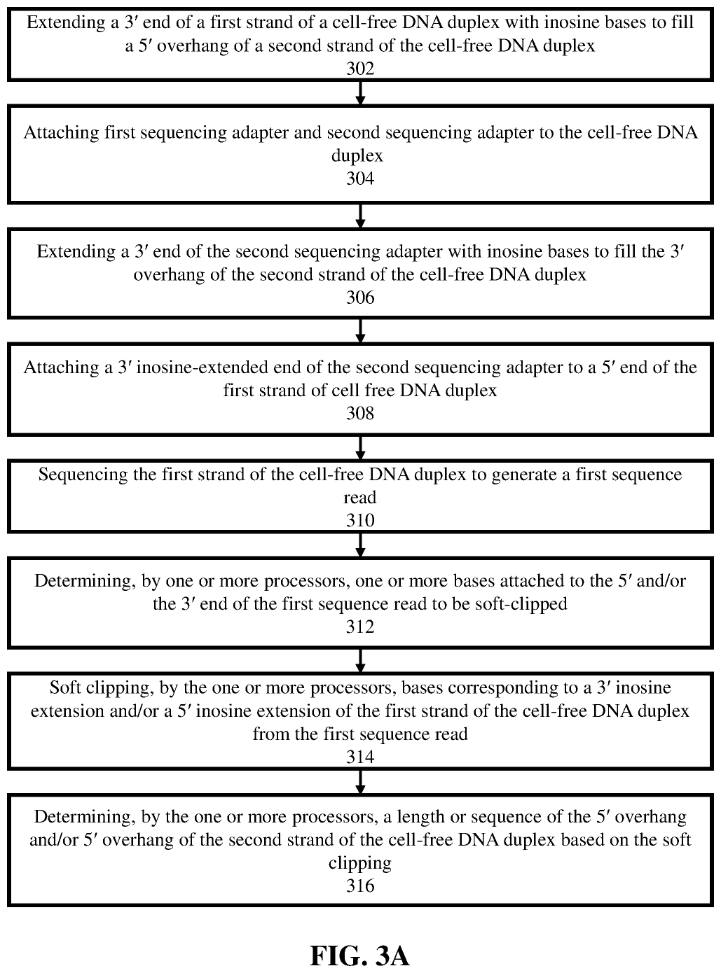

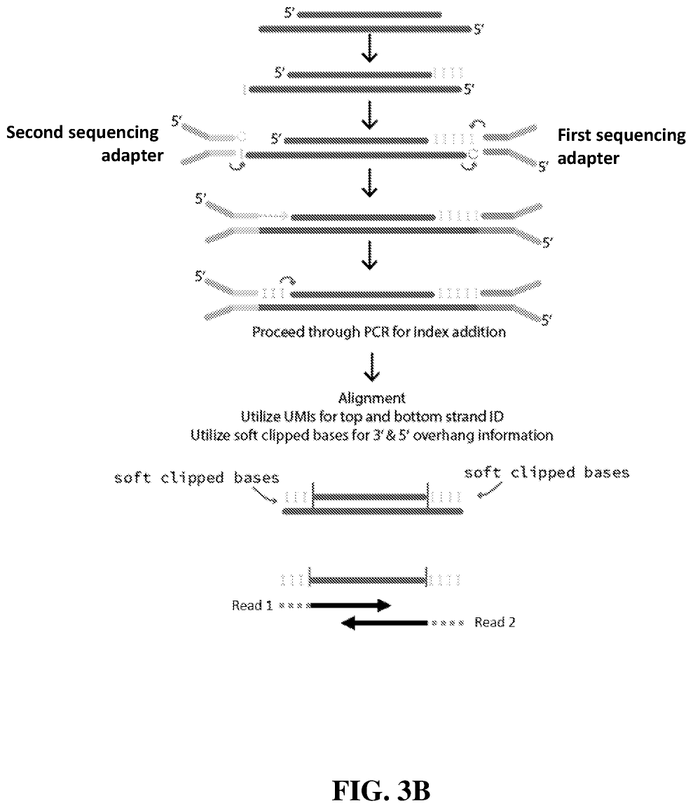

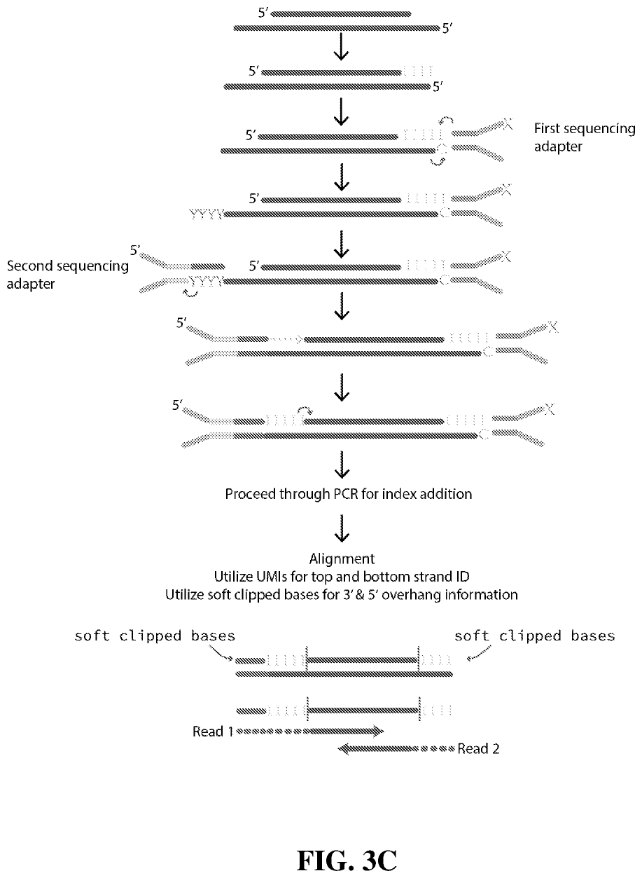

In some embodiments, the sequencing adapter is attached to the 3′ overhang of a first strand of a cell-free DNA duplex using one or more enzymes having terminal transferase and ligase activity, which extends the 3′ overhang and ligates the sequencing adapter to the 3′ overhang extension. This may be performed, for example, using an Adaptase™ module from xGen™, which includes enzymes with terminal transferase and ligase activity. The terminal transferase can generate a 3′ tail, which may be referred to as an “adaptase tail” or “AdT”. In some implementations, the 3′ tail provided by the terminal transferase activity includes a plurality of bases of the same base type. The sequencing adapter includes a 3′ overhang that complements a base of the 3′ attached to the 3′ overhang of the DNA duplex molecule. This process is further shown in FIG. 3C.

At 204 of FIG. 2A, the method further includes extending a 3′ end of the sequencing adapter with inosine bases to fill the 3′ overhang of the first strand of the cell-free DNA duplex. In some embodiments, the inosine is a deoxyinosine (dI). At 206, the 3′ inosine-extended end of the sequencing adapter is attached (for example, by ligation) to a 5′ end of a second strand of the cell free DNA duplex. This results in the attached 3′ inosine-extended end of the sequence adapter providing a 5′ inosine extension of the second strand of the cell-free DNA duplex. That is, the inosine extension is between the sequencing adapter and the original second strand of the DNA duplex molecule.

FIG. 2B shows an exemplary method for determining cell-free DNA topology, such as for determining a 3′ overhang length of a cell-free DNA duplex, which builds on the method for making the sequencing construct shown in FIG. 2A. At 208, the second strand of the cell-free DNA duplex is sequenced to generate a first sequence read. In some embodiments, the sequencing comprises next-generation sequencing (“NGS”). In some embodiments, the sequencing comprises paired-end sequencing. In some embodiments, the sequencing adapter or the second sequencing adapter comprise an amplification primer binding site, a flow cell adapter sequence, or a substrate adapter sequence. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencing is performed using a next generation sequencer.

At 210 of FIG. 2B, the method further includes determining, by one or more processors, one or more bases in the first sequence read to be soft-clipped. Determining the one or more bases in the first sequence read to be soft-clipped may include, for example, aligning the first sequence read to a reference sequence and identifying an unaligned portion of the first sequence read. The unaligned portion of the first sequence read can be associated with inosine bases in the extended first strand. Because these inosine bases are artificially generated, they do not align with the reference sequence or complementary strand, and therefore indicate an overhang in the complementary strand. These unaligned bases in the 5′ portion of the sequence read can therefore be identified for soft clipping.

In some embodiments, the method includes sequencing the second strand of the cell-free DNA duplex to generate a second sequence read. The one or more bases in the first sequence read to be soft clipped may be determined by aligning the first sequence (i.e., for the first strand) to the second sequence read (i.e., for the second strand) and identifying an unaligned portion of the first sequence read. Matching the first sequence read to the second sequence read may include the use of a UMI common between the first sequence read and the second sequence read. The unaligned 5′ portion of the sequence read may be identified for soft clipping.

At 212 of FIG. 2B, the method comprises soft clipping bases from the sequence reads, by the one or more processors, corresponding to the 5′ inosine extension of the second strand of the cell-free DNA duplex from the first sequence read. At 214, the method comprises determining, by the one or more processors, a length of the 3′ overhang of the first strand of the cell-free DNA duplex based on the soft clipping of the 5′ inosine extension of the second strand of cell-free DNA duplex. Based on the length of the 3′ overhang of the first strand of the cell-free DNA duplex and the sequence of the first strand of the cell-free DNA, the sequence of the 3′ overhang may be determined.

In some embodiments, there is provided a method of making a sequencing construct, comprising: attaching a sequencing adapter to a 3′ overhang of a first strand of a cell-free DNA duplex; extending a 3′ end of the sequencing adapter with inosine bases to fill the 3′ overhang of the first strand of the cell-free DNA duplex; and attaching a 3′ inosine-extended end of the sequencing adapter to a 5′ end of a second strand of the cell free DNA duplex, wherein the attached 3′ inosine-extended end provides a 5′ inosine extension of the second strand of the cell-free DNA duplex. In some embodiments, attaching the sequencing adapter to the 3′ overhang of the first strand of the cell-free DNA duplex comprises: extending the 3′ overhang of the first strand of the cell-free DNA duplex to provide a 3′ extension, wherein the sequencing adapter comprises a 3′ overhang that complements the 3′ extension of the first strand of the cell-free DNA duplex; and attaching the sequencing adapter to the 3′ extension of the first strand of the cell-free DNA duplex. In some embodiments, the 3′ overhang of the first strand of the cell-free DNA duplex is extended using nucleotide bases of the same base type.

In some embodiments, there is provided a method for determining cell-free DNA topology, comprising: making the sequencing construct described herein; sequencing the second strand of the cell-free DNA duplex to generate a first sequence read; determining, by one or more processors, one or more bases in the first sequence read to be soft-clipped; soft clipping, by the one or more processors, bases corresponding to the 5′ inosine extension of the second strand of the cell-free DNA duplex from the first sequence read; and determining, by the one or more processors, a length or sequence of the 3′ overhang of the first strand of the cell-free DNA duplex based on the soft clipping of the 5′ inosine extension of the second strand of cell-free DNA duplex. In some embodiments, the method comprises sequencing the first strand of the cell-free DNA duplex to generate a second sequence read; and determining, by one or more processors, one or more bases in the second sequence read to be soft-clipped.

In some embodiments, the method comprises: attaching a second sequencing adapter to a 3′ overhang of the second strand of a cell-free DNA duplex; extending a 3′ end of the second sequencing adapter with inosine bases to fill the 3′ overhang of the second strand of the cell-free DNA duplex; and attaching a 3′ inosine-extended end of the second sequencing adapter to a 5′ end of a first strand of the cell free DNA duplex, wherein the 3′ inosine-extended end provides a 5′ inosine extension of the first strand of the cell-free DNA duplex. In some embodiments, the method further comprises sequencing the first strand of the cell-free DNA duplex to generate a second sequence read; determining, by one or more processors, one or more bases attached to the 5′ end of the second sequence read to be soft-clipped; soft clipping, by the one or more processors, bases corresponding to a 5′ inosine extension of the first strand of the cell-free DNA duplex from the second sequence read; and determining, by the one or more processors, a length or sequence of the 3′ overhang of the second strand of the cell-free DNA duplex based on the soft clipping of the 5′ inosine extension of the first strand of the cell-free DNA duplex. In some embodiments, attaching the second sequencing adapter to the 3′ overhang of the second strand of a cell-free DNA duplex comprises attaching a single inosine to the 3′ overhang of the second strand of a cell-free DNA duplex and attaching the second sequencing adapter to the inosine. In some embodiments, the sequencing adapter comprises a 3′ cytosine overhang that complements to the single inosine attached to the 3′ overhang of the second strand of a cell-free DNA duplex.

In some embodiments, the method comprises amplifying the first strand and the second strand of the cell-free DNA duplex as described above. In some embodiments, the method comprises sequencing as described above. In some embodiments, determining the one or more bases in the first sequence read to be soft-clipped comprises aligning the first and/or sequence read as described above.

In some embodiments, the method further comprises detecting, by the one or more processors, a presence or absence of disease based on the length or sequence of the 3′ overhang of the first strand of the cell-free DNA duplex. In some embodiments, the method further comprises detecting, by the one or more processors, a presence or absence of disease based on the length or sequence of the 3′ overhang of the second strand of the cell-free DNA duplex. In some embodiments, the disease is cancer.

In some instances, the DNA duplex molecule includes both a 5′ overhang and a 3′ overhang. The method provided herein may be used generate a sequencing construct, and analyze such a construct, to determine both the 5′ overhang and 3′ overhang topology of the DNA duplex. FIG. 3A shows a method for determining DNA duplex (e.g., cfDNA) topology, such as for determining a 3′ overhang length and 5′ overhang length of a DNA duplex molecule (e.g., a cell-free DNA duplex molecule). As shown at 302, the method includes extending a 3′ end of a first strand of a DNA duplex with inosine bases to fill a 5′ overhang of a second strand of the cell-free DNA duplex. In some embodiments, the inosine is a deoxyinosine (dI). The 3′ end of the first strand of the DNA duplex molecule may be extended using a polymerase. Optionally, a polymerase capable of attaching a single nucleotide (for example, an inosine base) to the 3′ end (e.g., blunt end) of the DNA duplex molecule may be used to generate a single 3′ nucleotide overhang (e.g., a single 3′ inosine overhang). The polymerase may be, for example, a Taq polymerase. In another embodiment, a terminal transferase may be used to attach one or more (e.g., a plurality of nucleotide bases to the 3′ overhang end of the cfDNA duplex molecule. The attached bases may be, for example, canonical bases (e.g., A, C, T, or G). The 3′ overhang may increase attachment efficiency of the sequencing adapter, which may include a 3′ overhang that complements the 3′ overhang of the cfDNA molecule. In some embodiments, the attached bases are of the same nucleotide base type.

At 304, the method includes attaching a first sequencing adapter and a second sequencing adapter to the DNA duplex molecule. In some embodiments, attaching of a sequencing adapter comprises ligating of the sequencing adapter to the cell-free DNA duplex. The sequencing adapter may be, for example, a Y-shaped sequencing adapter. In some embodiments, the adapter may be a Y full length adapter (e.g., a Y-shaped adapter comprising an index sequence), a stubby adapter, or a hairpin adapter. The first sequencing adapter can be attached to the end of the DNA duplex molecule having the 5′ overhang, as shown in FIG. 3B and FIG. 3C. The first and sequence sequencing adapters may be attached to the DNA duplex molecule in the same reaction (for example, as shown in FIG. 3B) or in sequential reactions (for example, as shown in FIG. 3B). In the exemplary process shown in FIG. 3B, the first sequencing adapter includes a 3′ overhang that complements the 3′ tail on the first strand of the DNA duplex molecule. Similarly, the second sequencing adapter includes a 3′ overhang that complements the 3′ tail on the second strand of the DNA duplex molecule. As discussed above, a polymerase capable of attaching a single nucleotide (for example, an inosine base) to the 3′ end (e.g., blunt end) of the DNA duplex molecule may be used to generate a single 3′ nucleotide overhang (e.g., a single 3′ inosine overhang). Inosine is an analogue of guanine and, accordingly, preferentially pairs with cytosine bases (although it may pair with other canonical nucleotide bases (i.e., adenine, thymine, or guanine). If an inosine tail is included on the 3′ end(s), for example, the adapter(s) may include a 3′ overhang comprising a single nucleotide (e.g., cytosine base to preferentially complement the inosine base. The polymerase may be, for example, a Taq polymerase. In another embodiment, a terminal transferase may be used to attach one or more (e.g., a plurality of nucleotide bases to the 3′ overhang end of the cfDNA duplex molecule. The attached bases may be, for example, canonical bases (e.g., A, C, T, or G). The 3′ overhang may increase attachment efficiency of the sequencing adapter, which may include a 3′ overhang that complements the 3′ overhang of the cfDNA molecule. In some embodiments, the attached bases are of the same nucleotide base type.

In some implementations, the first and second sequencing adapters are attached to the DNA duplex molecule sequentially, for example attaching the second sequencing adapter to the DNA duplex molecule after attaching the first sequencing adapter to the DNA duplex molecule. The first sequencing adapter may be attached as described above. To attach the second sequencing adapter, an enzyme having terminal transferase may be used to extend the 3′ overhang of the second strand of the DNA molecule, as shown in FIG. 3C. This may be performed, for example, using an Adaptase™ module from xGen™, which includes enzymes with terminal transferase and ligase activity. The terminal transferase can generate a 3′ tail, which may be referred to as an “adaptase tail” or “AdT”. In some implementations, the 3′ tail provided by the terminal transferase activity includes a plurality of bases of the same base type. The sequencing adapter includes a 3′ overhang that complements a base of the 3′ attached to the 3′ overhang of the DNA duplex molecule

At 306 of FIG. 3A, the method further includes extending a 3′ end of the sequencing adapter with inosine bases to fill the 3′ overhang of the first strand of the cell-free DNA duplex. In some embodiments, the inosine is a deoxyinosine (dI). At 308, the 3′ inosine-extended end of the sequencing adapter is attached (for example, by ligation) to a 5′ end of a second strand of the cell free DNA duplex. This results in the attached 3′ inosine-extended end of the sequence adapter providing a 5′ inosine extension of the second strand of the cell-free DNA duplex. That is, the inosine extension is between the sequencing adapter and the original second strand of the DNA duplex molecule.

In some embodiments, sequencing construct is amplified, for example prior to sequencing. The amplifying process can allow for the attachment of a sample index to the cell-free DNA duplex. In some embodiments, however, a sample index is included in the sequencing adapter, making the incorporation of a sample index in a downstream amplification process unnecessary. In some embodiments, the amplifying comprises performing a polymerase chain reaction (PCR) amplification technique, a non-PCR amplification technique, or an isothermal amplification technique.

The first strand of the DNA duplex molecule is sequenced at 310 to generate a first sequence read. In some implementations, both the first strand and the second strand of the DNA duplex molecule are sequenced to generate a first sequence read and a second sequence read, respectively. In some embodiments, the sequencing comprises next-generation sequencing (“NGS”). In some embodiments, the sequencing comprises paired-end sequencing. In some embodiments, the sequencing adapter or the second sequencing adapter comprise an amplification primer binding site, a flow cell adapter sequence, or a substrate adapter sequence. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencing is performed using a next generation sequencer.

At 312, the method comprises determining, by one or more processors, one or more bases attached to the 5′ and/or the 3′ end of the first sequence read to be soft-clipped. As stated above, during amplification (e.g., PCR amplification), the inosine bases are converted to one or more nucleotides (e.g., A, T, G, C). During alignment, the nucleotides representing the inosine bases will not align (map) to the reference genome sequence and, therefore, will identify where the original cfDNA sequence (e.g., fragment) ends and where the inosine base sequence of the overhang gap begins. Therefore, determining the one or more bases in the first sequence read to be soft-clipped may include, for example, aligning the first sequence read to a reference sequence and identifying an unaligned portion of the first sequence read. The unaligned portion of the first sequence read (i.e., the unaligned nucleotides) can be associated with inosine bases in the extended first strand. Because these nucleotides representing the inosine bases are artificially generated, they do not align with the reference sequence or complementary strand, and therefore indicate an overhang in the complementary strand. These unaligned nucleotide bases in the 5′ portion and/or 3′ portion of the sequence read can therefore be identified for soft clipping.

In some embodiments, the method includes sequencing the second strand of the cell-free DNA duplex to generate a second sequence read. The one or more bases in the first sequence read to be soft clipped may be determined by aligning the first sequence (i.e., for the first strand) to the second sequence read (i.e., for the second strand) and identifying an unaligned portion of the first sequence read. Matching the first sequence read to the second sequence read may include the use of a UMI common between the first sequence read and the second sequence read. The unaligned 5′ portion and/or 3′ portion of the sequence read may be identified for soft clipping.

At 314, the method includes soft clipping, by the one or more processors, bases corresponding to a 3′ inosine extension and/or a 5′ inosine extension of the first strand of the cell-free DNA duplex from the first sequence read. At 316, the method comprises determining, by the one or more processors, a length or sequence of the 5′ overhang of the second strand of the cell-free DNA duplex based on the soft clipping of the 3′ inosine extension of the first strand of the cell-free DNA duplex and/or a length or sequence of the 3′ overhang of the second strand of the cell-free DNA duplex based on the soft clipping of the 5′ inosine extension of the first strand of cell-free DNA duplex.

In some embodiments, there is provided a method for determining cell-free DNA topology, comprising: extending a 3′ end of a first strand of a cell-free DNA duplex with inosine bases to fill a 5′ overhang of a second strand of the cell-free DNA duplex; attaching a sequencing adapter to the cell-free DNA duplex; sequencing the first strand of the cell-free DNA duplex to generate a first sequence read; determining, by one or more processors, one or more bases in the first sequence read to be soft-clipped; soft clipping, by the one or more processors, bases corresponding to a 3′ inosine extension of the first strand of the cell-free DNA duplex from the first sequence read; and determining, by the one or more processors, a length or sequence of the 5′ overhang of the second strand of the cell-free DNA duplex based on the soft clipping of the 3′ inosine extension of the first strand of the cell-free DNA duplex. In some embodiments, the method further comprises attaching a second sequencing adapter to a 3′ overhang of the second strand of the cell-free DNA duplex; extending a 3′ end of the second sequencing adapter with inosine bases to fill the 3′ overhang of the second strand of the cell-free DNA duplex; attaching a 3′ inosine-extended end of the second sequencing adapter to a 5′ end of the first strand of cell free DNA duplex, wherein the attached 3′ inosine-extended end provides a 5′ inosine extension of the first strand cell-free DNA duplex; determining, by one or more processors, one or more bases attached to the 5′ end of the first sequence read to be soft-clipped; soft clipping, by the one or more processors, bases corresponding to the 5′ inosine extension of the first strand of the cell-free DNA duplex from the first sequence read; and determining, by the one or more processors, a length or sequence of the 3′ overhang of the second strand of the cell-free DNA duplex based on the soft clipping of the 5′ inosine extension of the first strand of cell-free DNA duplex. In some embodiments, the method comprises extending a 3′ end of a second strand of the cell-free DNA duplex with inosine bases to fill a 5′ overhang of the first strand of the cell-free DNA duplex; attaching the second sequencing adapter to the cell-free DNA duplex; soft clipping, by the one or more processors, bases corresponding to a 3′ inosine extension of the second strand of the cell-free DNA duplex from the second sequence read; and determining, by the one or more processors, a length or sequence of the 5′ overhang of the first strand of the cell-free DNA duplex based on the soft clipping of the 3′ inosine extension of the second strand of the cell-free DNA duplex.

In some embodiments, attaching the second sequencing adapter to the 3′ overhang of the second strand of the cell-free DNA duplex comprises: extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and attaching the second sequencing adapter to the 3′ extension of the second strand of the cell-free DNA duplex. In some embodiments, the 3′ overhang of the second strand of the cell-free DNA duplex is extended using nucleotide bases of the same base type.

In some embodiments, the method further comprises detecting, by the one or more processors, a presence or absence of a disease based on the length or sequence of the 5′ overhang of the second strand of the cell-free DNA duplex. In some embodiments, the method further comprises detecting, by the one or more processors, a presence or absence of a disease based on the length or sequence of 3′ overhang of the second strand of the cell-free DNA duplex. In some embodiments, the method further comprises detecting, by the one or more processors, a presence or absence of a disease based on the length or sequence of 5′ overhang of the first strand of the cell-free DNA duplex. In some embodiments, the method further comprises detecting, by the one or more processors, a presence or absence of disease based on the length or sequence of the 3′ overhang of the first strand of the cell-free DNA duplex. In some embodiments, the method further comprises detecting, by the one or more processors, a presence or absence of a disease based on the length or sequence of the 3′ overhang of the second strand of the cell-free DNA duplex. In some embodiments, the disease is a cancer.

In some embodiments, the method comprises amplifying the first strand and the second strand of the cell-free DNA duplex as described above. In some embodiments, the method comprises sequencing as described above. In some embodiments, determining the one or more bases in the first sequence read to be soft-clipped comprises aligning the first and/or second sequence read as described above.

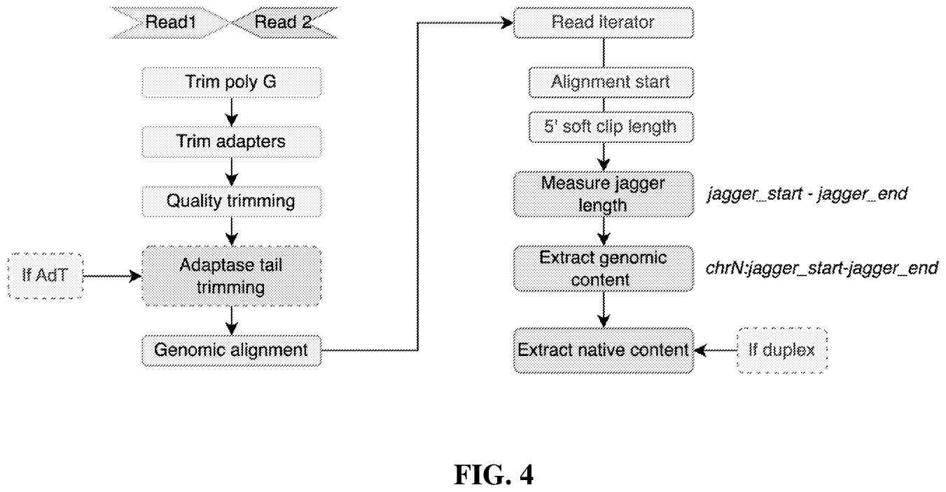

FIG. 4 shows process that is used to determine cfDNA topology, according to some embodiments. Sequence reads obtained by the methods described herein are received at one or more processors. In the process shown in FIG. 4, Read 1 and Read 2 represent reads sequenced from both ends of a single strand of the duplex. The sequence reads are subjected to pre-processing prior to further analysis, which may include, for example, trimming poly-G regions (e.g., dark cycles), adapter sequences, and/or portions of the sequence read considered to be low quality (for example, based on a sequencing quality score below a predetermined threshold, such as a Q score of Q10 or lower, Q20 or lower, or Q30 or lower). If the sequencing read includes an adaptase tail (i.e., a 3′ terminal tail added by a terminal transferase), this adaptase tail may be trimmed. The resulting sequencing read (i.e., following pre-processing) may be further analyzed to determine one or more bases in the sequencing read to be soft clipped, for example by aligning the sequence read to a reference sequence. Based on the alignment, one or more bases may be identified for soft clipping, and the soft clipped length is measured (i.e., the “jagger” length). This length is indicative of the overhang in the complementary strand. Genomic content of the complementary strand may be extracted to provide the sequence of the overhang.

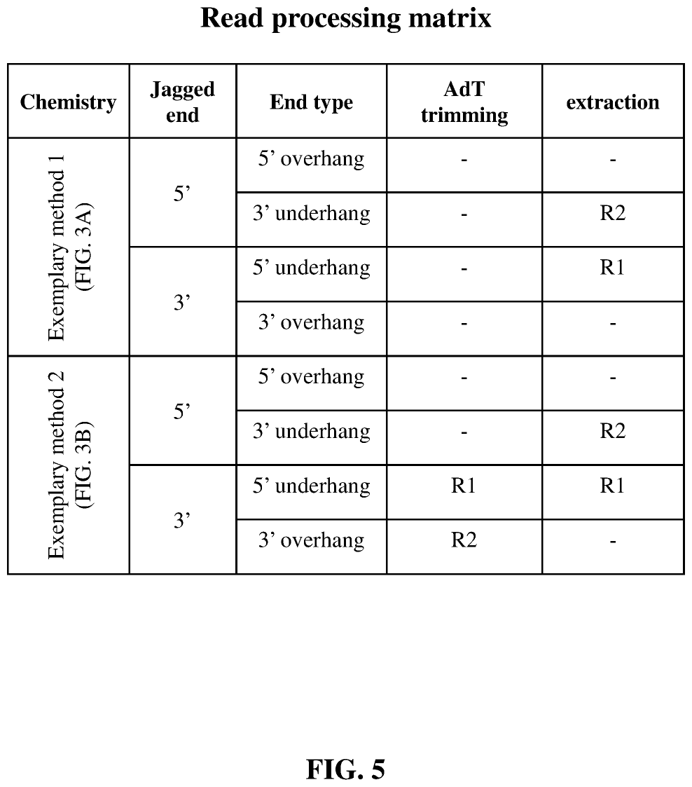

FIG. 5 shows an exemplary read processing matrix for extracting data from paired-end sequencing, according to some embodiments, including those sequence constructs and analytical methods shown in FIG. 3A and FIG. 3B.

In some implementations of any of the methods described, herein, the DNA duplex molecule is obtained from an individual. The method can further include generating a DNA duplex molecule overhang profile for the individual comprising information about overhangs for a plurality of DNA duplex molecules. For example, the information can include, for a plurality of DNA duplex molecules, the 3′ overhang length for the first strand of the DNA duplex molecule, a 5′ overhang length for the first strand of DNA duplex molecule, a 3′ overhang length for the second strand of the DNA duplex molecule, and/or a 5′ overhang length for the second strand of the DNA duplex molecule. In some implementations, the information comprises a sequence of a 5′ overhang or a 3′ overhang. In some implementations, the information comprises (1) a ratio of the a 5′ overhang length for the first strand of DNA duplex molecule to a 3′ overhang length for the first strand of DNA duplex molecule, (2) a ratio of the a 5′ overhang length for the first strand of DNA duplex molecule to a 3′ overhang length for the second strand of DNA duplex molecule, (3) a ratio of the a 5′ overhang length for the first strand of DNA duplex molecule to a 5′ overhang length for the second strand of DNA duplex molecule, (4) a ratio of the a 3′ overhang length for the first strand of DNA duplex molecule to a 5′ overhang length for the second strand of DNA duplex molecule, or (5) a ratio of the a 3′ overhang length for the first strand of DNA duplex molecule to a 3′ overhang length for the second strand of DNA duplex molecule. In some implementations, the information comprises a ratio of (1) a 5′ overhang length for the first strand of DNA duplex molecule, a 3′ overhang length for the first strand of DNA duplex molecule, a 5′ overhang length for the second strand of DNA duplex molecule, a 3′ overhang length for the second strand of DNA duplex molecule to (2) a duplex length.

The method may further include comparing, for example using one or more processors, the DNA duplex molecule overhang profile to a reference DNA duplex molecule overhang profile. In some implementations, the reference DNA duplex molecule overhang profile is based on DNA duplex molecules from a normal sample, a plurality of normal samples, or a synthetically generated normal sample. In some implementations, the reference DNA duplex molecule overhang profile is based on DNA duplex molecules from a sample obtained from an individual with cancer or a plurality of individuals with cancer. In some implementations, the reference DNA duplex molecule overhang profile is based on DNA duplex molecules from a sample obtained from an individual with an abnormal fetus or a plurality of individuals with an abnormal fetus. In some implementations, the reference DNA duplex molecule overhang profile is based on DNA duplex molecules from a sample obtained from an individual that received a stable transplant or a plurality of individuals that received a stable transplant. In some implementations, the reference DNA duplex molecule overhang profile is based on DNA duplex molecules from a match normal sample obtained from the individual. In some implementations, the reference DNA duplex molecule overhang profile is based on DNA duplex molecules from a prior sample obtained from the individual. This prior sample may be a normal baseline or a disease state baseline (i.e., a DNA duplex molecule overhang profile representing a prior state of a disease state).

In some embodiments, the method described herein further comprises generating, by one or more processors, a report indicating the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex, or other DNA duplex molecule overhang profile information. In some embodiments, the method further comprises transmitting the report to a healthcare provider. In some embodiments, the report is transmitted via a computer network, a peer-to-peer connection or to an application programming interface (API).

In some embodiments, the method further comprises generating a genomic profile for the subject comprising the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test.

The DNA topological information, such as 3′ overhang and/or 5′ overhang length and/or sequence information determined according to the methods described herein, may be used to detect the presence, likely presence, or absence of a disease, for example cancer. This is particularly useful, for example, in determining the presence or absence of disease in an early-stage cancer patient or a cancer patient having low cancer levels or detecting the recurrence of disease. For example, in some implementations the individual has a circulating tumor DNA (ctDNA) content of less than 1%, less than 0.8%, less than 0.5%, less than 0.3%, less than 0.1%, or less than 0.05%.

### Samples

The disclosed methods and systems may be used with any of a variety of samples (also referred to herein as specimens) comprising nucleic acids (e.g., DNA) that are collected from a subject (e.g., a patient). Examples of a sample include, but are not limited to, a liquid biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or sample derived therefrom).

In some embodiments, the sample may be collected by needle biopsy, fine needle aspiration, collection cup or tube, oral swab, nasal swab, vaginal swab or a cytology smear, etc.

In some embodiments, the sample is a liquid biopsy sample, and may comprise, e.g., whole blood, blood plasma, blood serum, urine, stool, sputum, saliva, or cerebrospinal fluid. In some embodiments, the sample may be a liquid biopsy sample and may comprise circulating tumor cells (CTCs). In some embodiments, the sample may be a liquid biopsy sample and may comprise cell-free DNA (cfDNA), circulating tumor DNA (ctDNA), or any combination thereof.

In some embodiments, the disclosed methods may further comprise analyzing a primary control (e.g., a normal blood sample). In some embodiments, the disclosed methods may further comprise determining if a primary control is available and, if so, isolating a control nucleic acid (e.g., DNA) from said primary control. In some embodiments, the sample may comprise any normal control if no primary control is available. In some embodiments, the method includes evaluating a sample, e.g., a normal sample using the methods described herein. In some embodiments, the disclosed methods may further comprise determining that no primary control is available, and marking said sample for analysis without a matched control.

In some embodiments, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some embodiments, cell-free or circulating tumor DNA is extracted from the liquid sample. In some embodiments, a sample with low nucleated cellularity may require more, e.g., greater volume for DNA extraction.

In some embodiments, the method for determining cell-free DNA topology further comprises obtaining the cell-free DNA duplex from a subject. In some embodiments, the cell-free DNA duplex is obtained from a liquid biopsy sample. In some embodiments, the sample is a liquid biopsy sample and comprises blood, plasma, cerebrospinal fluid, sputum, stool, urine, or saliva. In some embodiments, the cell-free DNA duplex is a circulating tumor DNA (ctDNA) duplex.

### Subjects

In some embodiments, the sample is obtained (e.g., collected) from a subject (e.g., a human patient) with a condition or disease (e.g., a hyperproliferative disease (such as cancer) or a non-cancer indication) or suspected of having the condition or disease. In some embodiments, the hyperproliferative disease is a cancer. In some embodiments, the cancer is a solid tumor or a metastatic form thereof. In some embodiments, the cancer is a hematological cancer, e.g., a leukemia or lymphoma.

In some embodiments, the subject has a cancer or is at risk of having a cancer. For example, in some embodiments, the subject has a genetic predisposition to a cancer (e.g., having a genetic mutation that increases their baseline risk for developing a cancer). In some embodiments, the subject has been exposed to an environmental perturbation (e.g., radiation or a chemical) that increases their risk for developing a cancer. In some embodiments, the subject is in need of being monitored for development of a cancer. In some embodiments, the subject is in need of being monitored for cancer progression or regression, e.g., after being treated with an anti-cancer therapy (or anti-cancer treatment). In some embodiments, the subject is in need of being monitored for relapse of cancer. In some embodiments, the subject is in need of being monitored for minimum residual disease (MRD). In some embodiments, the subject has been or is being treated for cancer. In some embodiments, the subject has not been treated with an anti-cancer therapy (or anti-cancer treatment).

In some embodiments, the subject (e.g., a patient) is being treated, or has been previously treated, with one or more anti-cancer therapies. In some embodiments, e.g., for a patient who has been previously treated with a targeted anti-cancer therapy, a post-targeted therapy sample (e.g., specimen) is obtained (e.g., collected). In some embodiments, the post-targeted therapy sample is a sample obtained after the completion of the targeted therapy. In some embodiments, the one or more anti-cancer therapies (or anti-cancer treatments) can include, but are not limited to, surgery (e.g., surgical resection), radiation therapy, or chemotherapy and mixtures thereof or the like.

In some embodiments, the patient has not been previously treated with an anti-cancer therapy. In some embodiments, e.g., for a patient who has not been previously treated with a targeted anti-cancer therapy, the sample comprises a liquid biopsy, e.g., an original liquid biopsy, or a liquid biopsy following recurrence.

In some embodiments, the method for determining cell-free DNA topology further comprises obtaining the cell-free DNA duplex from a subject. In some embodiments, the cell-free DNA duplex is obtained from a subject suspected of having cancer or determined to have cancer. In some embodiments, the cell-free DNA duplex is obtained from a liquid biopsy sample. In some embodiments, the sample is a liquid biopsy sample and comprises blood, plasma, cerebrospinal fluid, sputum, stool, urine, or saliva. In some embodiments, the cell-free DNA duplex is a circulating tumor DNA (ctDNA) duplex. In some embodiments, the method further comprises treating the subject with an anti-cancer therapy.

### Nucleic Acid Extraction and Processing

DNA may be extracted from liquid biopsy samples, including but not limited to blood, plasma, cerebrospinal fluid, sputum, stool, urine, or saliva or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)).

A typical DNA extraction procedure, for example, comprises (i) collection of the fluid sample from which DNA is to be extracted, (ii) treatment of the fluid sample with a concentrated salt solution to precipitate proteins, lipids, and RNA, followed by centrifugation to separate out the precipitated proteins, lipids, and RNA, and (iii) purification of DNA from the supernatant to remove detergents, proteins, salts, or other reagents used during previous steps. The DNA sample optionally may be further treated with an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water.

### Systems and Storage Media

Further described herein are systems and computer-readable storage media that include instructors for causing the system to perform the methods described herein. An exemplary system includes, for example, one or more processors; and a memory communicatively coupled to the one or more processors and configured to store instructions that, when executed by the one or more processors, cause the system to: receive a first sequence read obtained by extending a 3′ end of a first strand of a cell-free DNA duplex with inosine bases to fill a 5′ overhang of a second strand of the cell-free DNA duplex; attaching a sequencing adapter to the cell-free DNA duplex; and sequencing the first strand of the cell-free DNA duplex to generate a first sequence read; determine one or more bases in the first sequence read to be soft-clipped; soft clip bases corresponding to a 3′ inosine extension of the first strand of the cell-free DNA duplex from the first sequence read; and determine a length or sequence of the 5′ overhang of the second strand of the cell-free DNA duplex based on the soft clipping of the 3′ inosine extension of the first strand of the cell-free DNA duplex.

In another example, a system includes one or more processors; and a memory communicatively coupled to the one or more processors and configured to store instructions that, when executed by the one or more processors, cause the system to: receive a first sequence read obtained by attaching a sequencing adapter to a 3′ overhang of a first strand of a cell-free DNA duplex; extending a 3′ end of the sequencing adapter with inosine bases to fill the 3′ overhang of the first strand of the cell-free DNA duplex; and attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex; determine one or more bases in the first sequence read to be soft-clipped; soft clip bases corresponding to the 5′ inosine extension of the second strand of the cell-free DNA duplex from the first sequence read; and determine a length or sequence of the 3′ overhang of the first strand of the cell-free DNA duplex based on the soft clipping of the 5′ inosine extension of the second strand of cell-free DNA duplex.

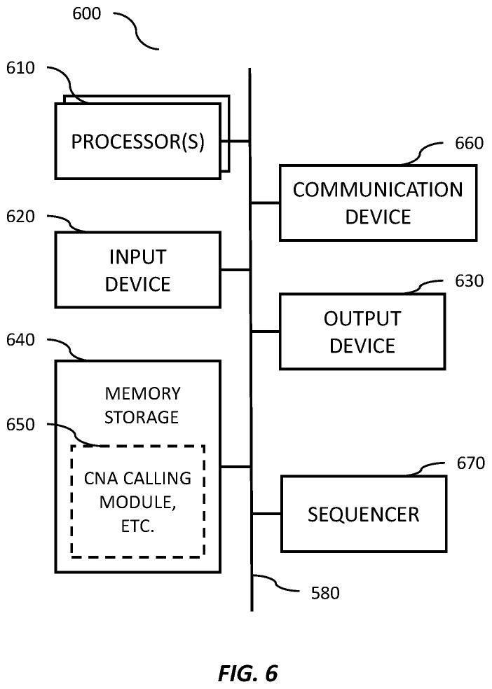

FIG. 6 illustrates an example of a computing device or system in accordance with one embodiment. Device 600 can be a host computer connected to a network. Device 900 can be a client computer or a server. As shown in FIG. 6, device 600 can be any suitable type of microprocessor-based device, such as a personal computer, workstation, server or handheld computing device (portable electronic device) such as a phone or tablet. The device can include, for example, one or more processor(s) 610, input devices 620, output devices 630, memory or storage devices 640, communication devices 660, and nucleic acid sequencers 670. Software 650 residing in memory or storage device 640 may comprise, e.g., an operating system as well as software for executing the methods described herein. Input device 620 and output device 630 can generally correspond to those described herein, and can either be connectable or integrated with the computer.

Input device 620 can be any suitable device that provides input, such as a touch screen, keyboard or keypad, mouse, or voice-recognition device. Output device 630 can be any suitable device that provides output, such as a touch screen, haptics device, or speaker.

Storage 640 can be any suitable device that provides storage (e.g., an electrical, magnetic or optical memory including a RAM (volatile and non-volatile), cache, hard drive, or removable storage disk). Communication device 660 can include any suitable device capable of transmitting and receiving signals over a network, such as a network interface chip or device. The components of the computer can be connected in any suitable manner, such as via a wired media (e.g., a physical system bus 680, Ethernet connection, or any other wire transfer technology) or wirelessly (e.g., Bluetooth®, Wi-Fi®, or any other wireless technology).

Software module 650, which can be stored as executable instructions in storage 640 and executed by processor(s) 610, can include, for example, an operating system and/or the processes that embody the functionality of the methods of the present disclosure (e.g., as embodied in the devices as described herein).

Software module 650 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 640, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

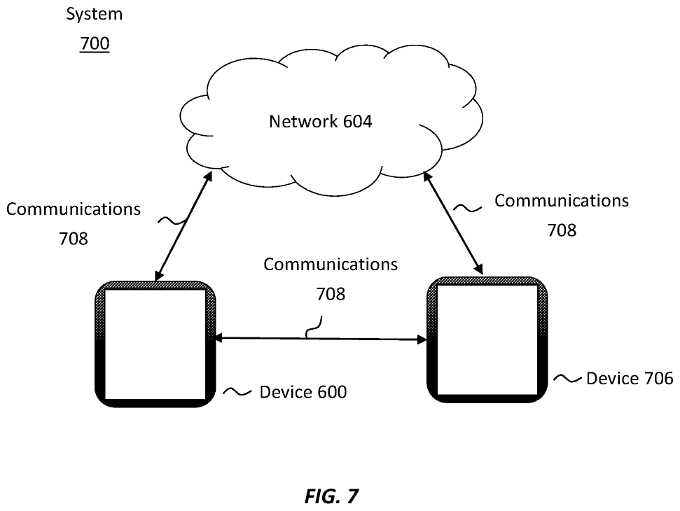

Device 600 may be connected to a network (e.g., network 704, as shown in FIG. 7 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 600 can be implemented using any operating system, e.g., an operating system suitable for operating on the network. Software module 650 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 610.

Device 600 can further include a sequencer 670, which can be any suitable nucleic acid sequencing instrument.

FIG. 7 illustrates an example of a computing system in accordance with one embodiment. In system 700, device 600 (e.g., as described above and illustrated in FIG. 6) is connected to network 704, which is also connected to device 706. In some embodiments, device 706 is a sequencer. Exemplary sequencers can include, without limitation, Roche/454′s Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 600 and 706 may communicate, e.g., using suitable communication interfaces via network 704, such as a Local Area Network (LAN), Virtual Private Network (VPN), or the Internet. In some embodiments, network 704 can be, for example, the Internet, an intranet, a virtual private network, a cloud network, a wired network, or a wireless network. Devices 600 and 706 may communicate, in part or in whole, via wireless or hardwired communications, such as Ethernet, IEEE 802.11b wireless, or the like. Additionally, devices 600 and 706 may communicate, e.g., using suitable communication interfaces, via a second network, such as a mobile/cellular network. Communication between devices 600 and 706 may further include or communicate with various servers such as a mail server, mobile server, media server, telephone server, and the like. In some embodiments, Devices 600 and 706 can communicate directly (instead of, or in addition to, communicating via network 704), e.g., via wireless or hardwired communications, such as Ethernet, IEEE 802.11b wireless, or the like. In some embodiments, devices 900 and 1006 communicate via communications 708, which can be a direct connection or can occur via a network (e.g., network 704).

One or all of devices 600 and 706 generally include logic (e.g., http web server logic) or are programmed to format data, accessed from local or remote databases or other sources of data and content, for providing and/or receiving information via network 704 according to various examples described herein.

### EXEMPLARY EMBODIMENTS

The following embodiments are exemplary and are not intended to limit the scope of any invention described herein. Exemplary embodiments include, but are not limited to:

Embodiment 1. A method for determining cell-free DNA topology, comprising:
extending a 3′ end of a first strand of a cell-free DNA duplex with inosine bases to fill a 5′ overhang of a second strand of the cell-free DNA duplex;
attaching a sequencing adapter to the cell-free DNA duplex;
sequencing the first strand of the cell-free DNA duplex to generate a first sequence read;
determining, by one or more processors, one or more bases in the first sequence read to be soft-clipped;
soft clipping, by the one or more processors, bases corresponding to a 3′ inosine extension of the first strand of the cell-free DNA duplex from the first sequence read; and
determining, by the one or more processors, a length or sequence of the 5′ overhang of the second strand of the cell-free DNA duplex based on the soft clipping of the 3′ inosine extension of the first strand of the cell-free DNA duplex.

Embodiment 2. The method of claim 1, further comprising detecting, by the one or more processors, a presence or absence of a disease based on the length or sequence of the 5′ overhang of the second strand of the cell-free DNA duplex.

Embodiment 3. The method of claim 2, wherein the disease is a cancer.

Embodiment 4. The method of any one of claims 1-3, further comprising:
attaching a second sequencing adapter to a 3′ overhang of the second strand of the cell-free DNA duplex;
extending a 3′ end of the second sequencing adapter with inosine bases to fill the 3′ overhang of the second strand of the cell-free DNA duplex;
attaching a 3′ inosine-extended end of the second sequencing adapter to a 5′ end of the first strand of cell-free DNA duplex, wherein the attached 3′ inosine-extended end provides a 5′ inosine extension of the first strand cell-free DNA duplex;
determining, by one or more processors, one or more bases attached to the 5′ end of the first sequence read to be soft-clipped;
soft clipping, by the one or more processors, bases corresponding to the 5′ inosine extension of the first strand of the cell-free DNA duplex from the first sequence read; and
determining, by the one or more processors, a length or sequence of the 3′ overhang of the second strand of the cell-free DNA duplex based on the soft clipping of the 5′ inosine extension of the first strand of cell-free DNA duplex.

Embodiment 5. The method of claim 4, comprising detecting, by the one or more processors, a presence or absence of a disease based on the length or sequence of 3′ overhang of the second strand of the cell-free DNA duplex.

Embodiment 6. The method of claim 4 or 5, wherein attaching the second sequencing adapter to the 3′ overhang of the second strand of the cell-free DNA duplex comprises:
extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and
attaching the second sequencing adapter to the 3′ extension of the second strand of the cell-free DNA duplex.

Embodiment 7. The method of claim 6, wherein the 3′ overhang of the second strand of the cell-free DNA duplex is extended using nucleotide bases of the same base type.

Embodiment 8. The method of any one of claims 1-7, wherein determining the one or more bases in the first sequence read to be soft-clipped comprises aligning the first sequence read to a reference sequence and identifying an unaligned portion of the first sequence read.

Embodiment 9. The method of any one of claims 1-8, further comprising:
sequencing the second strand of the cell-free DNA duplex to generate a second sequence read; and
determining, by one or more processors, one or more bases in the second sequence read to be soft-clipped.

Embodiment 10. The method of claim 9, wherein determining the one or more bases in the second sequence read to be soft-clipped comprises aligning the second sequence to a reference sequence and identifying an unaligned portion of the second sequence read.

Embodiment 11. The method of claim 9 or 10, wherein the first sequence read and the second sequence read are associated through a unique molecular identifier (UMI).

Embodiment 12. The method of claim 11, wherein determining the one or more bases in the first sequence read to be soft-clipped comprises aligning the first sequence to the second sequence read and identifying an unaligned portion of the first sequence read.

Embodiment 13. The method of claim 11 or 12, wherein determining the one or more bases in the second sequence read to be soft-clipped comprises aligning the second sequence to the first sequence read and identifying an unaligned portion of the second sequence read.

Embodiment 14. The method of any one of claims 9-13, comprising:
extending a 3′ end of a second strand of the cell-free DNA duplex with inosine bases to fill a 5′ overhang of the first strand of the cell-free DNA duplex;
attaching the second sequencing adapter to the cell-free DNA duplex;
soft clipping, by the one or more processors, bases corresponding to a 3′ inosine extension of the second strand of the cell-free DNA duplex from the second sequence read; and
determining, by the one or more processors, a length or sequence of the 5′ overhang of the first strand of the cell-free DNA duplex based on the soft clipping of the 3′ inosine extension of the second strand of the cell-free DNA duplex.

Embodiment 15. The method of claim 14, comprising detecting, by the one or more processors, a presence or absence of a disease based on the length or sequence of 5′ overhang of the first strand of the cell-free DNA duplex.

Embodiment 16. The method of any one of claims 1-15, wherein extending the 3′ end of the first strand of the cell-free DNA duplex comprises forming a single 3′ inosine overhang.

Embodiment 17. The method of claim 16, wherein the sequencing adapter comprises a 3′ cytosine overhang that complements to the 3′ inosine overhang.

Embodiment 18. The method of any one of claims 1-17, comprising amplifying the cell-free DNA duplex to attach a sample index to the cell-free DNA duplex.

Embodiment 19. The method of any one of claims 1-18, wherein the sequencing adapter or the second sequencing adapter comprises a sample index.

Embodiment 20. The method of any one of claims 11-19, wherein the sequencing adapter or the second sequencing adapter comprises the UMI.

Embodiment 21. The method of any one of claims 1-20, wherein the sequencing adapter or the second sequencing adapter is a Y-shaped sequencing adapter.

Embodiment 22. A method of making a sequencing construct, comprising:
attaching a sequencing adapter to a 3′ overhang of a first strand of a cell-free DNA duplex;
extending a 3′ end of the sequencing adapter with inosine bases to fill the 3′ overhang of the first strand of the cell-free DNA duplex; and
attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex.

Embodiment 23. The method of claim 22, wherein attaching the sequencing adapter to the 3′ overhang of the first strand of the cell-free DNA duplex comprises:
extending the 3′ overhang of the first strand of the cell-free DNA duplex to provide a 3′ extension, wherein the sequencing adapter comprises a 3′ overhang that complements the 3′ extension of the first strand of the cell-free DNA duplex; and
attaching the sequencing adapter to the 3′ extension of the first strand of the cell-free DNA duplex.

Embodiment 24 The method of claim 23, wherein the 3′ overhang of the first strand of the cell-free DNA duplex is extended using nucleotide bases of the same base type.

Embodiment 25. A method for determining cell-free DNA topology, comprising,
making the sequencing construct according to the method of any one of claims 22-24;
sequencing the second strand of the cell-free DNA duplex to generate a first sequence read;
determining, by one or more processors, one or more bases in the first sequence read to be soft-clipped;
soft clipping, by the one or more processors, bases corresponding to the 5′ inosine extension of the second strand of the cell-free DNA duplex from the first sequence read; and
determining, by the one or more processors, a length or sequence of the 3′ overhang of the first strand of the cell-free DNA duplex based on the soft clipping of the 5′ inosine extension of the second strand of cell-free DNA duplex.

Embodiment 26. The method of claim 25, further comprising detecting, by the one or more processors, a presence or absence of disease based on the length or sequence of the 3′ overhang of the first strand of the cell-free DNA duplex.

Embodiment 27. The method of claim 26, wherein the disease is cancer.

Embodiment 28. The method of any one of claims 25-27, wherein determining the one or more bases in the first sequence read to be soft-clipped comprises aligning the first sequence read to a reference sequence and identifying an unaligned portion of the first sequence read.

Embodiment 29. The method of any one of claims 25-28, further comprising:
sequencing the first strand of the cell-free DNA duplex to generate a second sequence read; and
determining, by one or more processors, one or more bases in the second sequence read to be soft-clipped.

Embodiment 30. The method of claim 29, wherein determining the one or more bases in the second sequence read to be soft-clipped comprises aligning the second sequence to a reference sequence and identifying an unaligned portion of the second sequence read.

Embodiment 31. The method of claim 29 or 30, wherein the first sequence read and the second sequence read are associated through a unique molecular identifier (UMI).

Embodiment 32. The method of claim 21, wherein determining the one or more bases in the first sequence read to be soft-clipped comprises aligning the first sequence to the second sequence read and identifying an unaligned portion of the first sequence read.

Embodiment 33. The method of claim 11 or 12, wherein determining the one or more bases in the second sequence read to be soft-clipped comprises aligning the second sequence to the first sequence read and identifying an unaligned portion of the second sequence read.

Embodiment 34. The method of any one of claims 22-33, comprising:
attaching a second sequencing adapter to a 3′ overhang of the second strand of a cell-free DNA duplex;
extending a 3′ end of the second sequencing adapter with inosine bases to fill the 3′ overhang of the second strand of the cell-free DNA duplex; and
attaching a 3′ inosine-extended end of the second sequencing adapter to a 5′ end of a first strand of the cell-free DNA duplex, wherein the 3′ inosine-extended end provides a 5′ inosine extension of the first strand of the cell-free DNA duplex.

Embodiment 35. The method of claim 34, further comprising:
sequencing the first strand of the cell-free DNA duplex to generate a second sequence read;
determining, by one or more processors, one or more bases attached to the 5′ end of the second sequence read to be soft-clipped;
soft clipping, by the one or more processors, bases corresponding to a 5′ inosine extension of the first strand of the cell-free DNA duplex from the second sequence read; and
determining, by the one or more processors, a length or sequence of the 3′ overhang of the second strand of the cell-free DNA duplex based on the soft clipping of the 5′ inosine extension of the first strand of the cell-free DNA duplex.

Embodiment 36. The method of claim 35, further comprising detecting, by the one or more processors, a presence or absence of disease based on the length or sequence of the 3′ overhang of the second strand of the cell-free DNA duplex.

Embodiment 37. The method of any one of claims 34-36, wherein attaching the second sequencing adapter to the 3′ overhang of the second strand of a cell-free DNA duplex comprises attaching a single inosine to the 3′ overhang of the second strand of a cell-free DNA duplex and attaching the second sequencing adapter to the single inosine.

Embodiment 38. The method of claim 37, wherein the sequencing adapter comprises a 3′ cytosine overhang that complements to the single inosine attached to the 3′ overhang of the second strand of a cell-free DNA duplex.

Embodiment 39. The method of any one of claims 35-38, wherein determining the one or more bases attached to the 5′ end of the second sequence read to be soft-clipped comprises aligning the second sequence read to a reference sequence and identifying an unaligned portion of the second sequence read.

Embodiment 40. The method of any one of claims 35-39, first sequence read and the second sequence read are associated through a unique molecular identifier (UMI).

Embodiment 41. The method of claim 40, wherein determining the one or more bases attached to the 5′ end of the second sequence read to be soft-clipped comprises aligning the second sequence read to the first sequence read and identifying an unaligned portion of the second sequence read.

Embodiment 42. The method of claim 40 or 41, wherein determining the one or more bases attached to the 5′ end of the first sequence read to be soft-clipped comprises aligning the first sequence read to the second sequence read and identifying an unaligned portion of the first sequence read.

Embodiment 43. The method of any one of claims 22-42, comprising amplifying the cell-free DNA duplex to attach a sample index to the cell-free DNA duplex.

Embodiment 44. The method of any one of claims 22-42, wherein the sequencing adapter or the second sequencing adapter comprises a sample index.

Embodiment 45. The method of any one of claims 40-44, wherein the sequencing adapter or the second sequencing adapter comprises the UMI.

Embodiment 46. The method of any one of claims 22-45, wherein the sequencing adapter or the second sequencing adapter is a Y-shaped sequencing adapter.

Embodiment 47. The method of any one of claims 1-46, wherein the cell-free DNA duplex is obtained from a subject suspected of having cancer or determined to have cancer.

Embodiment 48. The method of claim 47, further comprising treating the subject with an anti-cancer therapy.

Embodiment 49. The method of any one of claims 1-48, further comprising obtaining the cell-free DNA duplex from a subject.

Embodiment 50. The method of any one of claims 1-49, wherein the cell-free DNA duplex is obtained from a liquid biopsy sample.

Embodiment 51. The method of claim 50, wherein the sample is a liquid biopsy sample and comprises blood, plasma, cerebrospinal fluid, sputum, stool, urine, or saliva.

Embodiment 52. The method of any one of claims 1-51, wherein the cell-free DNA duplex is a circulating tumor DNA (ctDNA) duplex.

Embodiment 53. The method of any one of claims 1-52, wherein the sequencing adapter or the second sequencing adapter comprise an amplification primer binding site, a flow cell adapter sequence, or a substrate adapter sequence.

Embodiment 54. The method of any one of claims 1-53, further comprising amplifying the first strand and the second strand of the cell-free DNA duplex.

Embodiment 55. The method of claim 54, wherein the amplifying comprises performing a polymerase chain reaction (PCR) amplification technique, a non-PCR amplification technique, or an isothermal amplification technique.

Embodiment 56. The method of any one of claims 1-55, wherein the sequencing comprises use of a massively parallel sequencing (MPS) technique, whole genome sequencing (WGS), whole exome sequencing, targeted sequencing, direct sequencing, or Sanger sequencing technique.

Embodiment 57. The method of claim 56, wherein the sequencing comprises massively parallel sequencing, and the massively parallel sequencing technique comprises next generation sequencing (NGS).

Embodiment 58. The method of any one of claims 1-57, wherein the sequencing is performed using a next generation sequencer.

Embodiment 59. The method of any one of claims 1-58, further comprising generating, by one or more processors, a report indicating the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex.

Embodiment 60. The method of claim 59, further comprising transmitting the report to a healthcare provider.

Embodiment 61. The method of claim 60, wherein the report is transmitted via a computer network or a peer-to-peer connection.

Embodiment 62. The method of any one of claims 1-61, further comprising generating a genomic profile for the subject comprising the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex.

Embodiment 63. The method of claim 62, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 64. The method of claim 62 or 63, wherein the genomic profile for the subject further comprises results from a nucleic acid sequencing-based test.

Embodiment 65. A system comprising:
one or more processors; and
a memory communicatively coupled to the one or more processors and configured to store instructions that, when executed by the one or more processors, cause the system to:
   receive a first sequence read obtained by extending a 3′ end of a first strand of a cell-free DNA duplex with inosine bases to fill a 5′ overhang of a second strand of the cell-free DNA duplex; attaching a sequencing adapter to the cell-free DNA duplex; and sequencing the first strand of the cell-free DNA duplex to generate a first sequence read;
   determine one or more bases in the first sequence read to be soft-clipped;
   soft clip bases corresponding to a 3′ inosine extension of the first strand of the cell-free DNA duplex from the first sequence read; and
   determine a length or sequence of the 5′ overhang of the second strand of the cell-free DNA duplex based on the soft clipping of the 3′ inosine extension of the first strand of the cell-free DNA duplex.

Embodiment 66. The system of claim 65, wherein the instructions further cause the system to detect a presence or absence of a disease based on the length or sequence of the 5′ overhang of the second strand of the cell-free DNA duplex.

Embodiment 67. The system of claim 66, wherein the disease is a cancer.

Embodiment 68. The system of any one of claims 65-67, wherein the first sequence read is further obtained by attaching a second sequencing adapter to a 3′ overhang of the second strand of the cell-free DNA duplex; extending a 3′ end of the second sequencing adapter with inosine bases to fill the 3′ overhang of the second strand of the cell-free DNA duplex; and attaching a 3′ inosine-extended end of the second sequencing adapter to a 5′ end of the first strand of cell-free DNA duplex, wherein the attached 3′ inosine-extended end provides a 5′ inosine extension of the first strand cell-free DNA duplex.

Embodiment 69. The system of claim 68, wherein the instructions, when executed by the one or more processors, further cause the system to:
soft clip bases corresponding to the 5′ inosine extension of the first strand of the cell-free DNA duplex from the first sequence read; and
determine a length or sequence of the 3′ overhang of the second strand of the cell-free DNA duplex based on the soft clipping of the 5′ inosine extension of the first strand of cell-free DNA duplex.

Embodiment 70. The system of claim 69, wherein the instructions further cause the system to detect a presence or absence of a disease based on the length or sequence of the 3′ overhang of the second strand of the cell-free DNA duplex.

Embodiment 71. The system of any one of claims 68-70, wherein attaching the second sequencing adapter to the 3′ overhang of the second strand of the cell-free DNA duplex comprises:
extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and
attaching the second sequencing adapter to the 3′ extension of the second strand of the cell-free DNA duplex.

Embodiment 72. The system of claim 71, wherein the 3′ overhang of the second strand of the cell-free DNA duplex is extended using nucleotide bases of the same base type.

Embodiment 73. The system of any one of claims 65-72, wherein the one or more bases in the first sequence read is determined to be soft-clipped by a method comprising aligning the first sequence read to a reference sequence and identifying an unaligned portion of the first sequence read.

Embodiment 74. The system of any one of claims 65-73, wherein the instructions, when executed by the one or more processors, further cause the system to:
receive a second sequence read obtained by sequencing the second strand of the cell-free DNA duplex, and
determine one or more bases in the second sequence read to be soft-clipped.

Embodiment 75. The system of claim 74, wherein the one or more bases in the second sequence read are determined to be soft-clipped according to a method comprising aligning the second sequence to a reference sequence and identifying an unaligned portion of the second sequence read.

Embodiment 76. The system of claim 74 or 75, wherein the first sequence read and the second sequence read are associated through a unique molecular identifier (UMI).

Embodiment 77. The system of claim 76, wherein the one or more bases in the first sequence read are determined to be soft-clipped according to a method comprising aligning the first sequence to the second sequence read and identifying an unaligned portion of the first sequence read.

Embodiment 78. The system of claim 76 or 77, wherein the one or more bases in the second sequence read are determined to be soft-clipped according to a method comprising aligning the second sequence to the first sequence read and identifying an unaligned portion of the second sequence read.

Embodiment 79. The system of any one of claims 74-78, wherein the second sequence read is further obtained by extending a 3′ end of a second strand of the cell-free DNA duplex with inosine bases to fill a 5′ overhang of the first strand of the cell-free DNA duplex; and attaching the second sequencing adapter to the cell-free DNA duplex.

Embodiment 80. The system of claim 79, wherein the instructions, when executed by the one or more processors, further cause the system to:
soft clip bases corresponding to a 3′ inosine extension of the second strand of the cell-free DNA duplex from the second sequence read; and
determine a length or sequence of the 5′ overhang of the first strand of the cell-free DNA duplex based on the soft clipping of the 3′ inosine extension of the second strand of the cell-free DNA duplex.

Embodiment 81. The system of any one of claims 65-80, wherein extending the 3′ end of the first strand of the cell-free DNA duplex comprises forming a 3′ single inosine overhang.

Embodiment 82. The system of claim 81, wherein the sequencing adapter comprises a 3′ cytosine overhang that complements to the 3′ inosine overhang.

Embodiment 83. A system comprising:
one or more processors; and
a memory communicatively coupled to the one or more processors and configured to store instructions that, when executed by the one or more processors, cause the system to:
   receive a first sequence read obtained by attaching a sequencing adapter to a 3′ overhang of a first strand of a cell-free DNA duplex; extending a 3′ end of the sequencing adapter with inosine bases to fill the 3′ overhang of the first strand of the cell-free DNA duplex; and attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex;
   determine one or more bases in the first sequence read to be soft-clipped;
   soft clip bases corresponding to the 5′ inosine extension of the second strand of the cell-free DNA duplex from the first sequence read; and
   determine a length or sequence of the 3′ overhang of the first strand of the cell-free DNA duplex based on the soft clipping of the 5′ inosine extension of the second strand of cell-free DNA duplex.

Embodiment 84. The system of claim 83, wherein the instructions further cause the system to detect a presence or absence of a disease based on the length or sequence of the 3′ overhang of the first strand of the cell-free DNA duplex.

Embodiment 85. The system of claim 84, wherein the disease is cancer.

Embodiment 86. The system of any one of claims 83-85, wherein attaching the second sequencing adapter to the 3′ overhang of the second strand of the cell-free DNA duplex comprises:
extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and
attaching the second sequencing adapter to the 3′ extension of the second strand of the cell-free DNA duplex.

Embodiment 87. The system of claim 86, wherein the 3′ overhang of the second strand of the cell-free DNA duplex is extended using nucleotide bases of the same base type.

Embodiment 88. The system of any one of claims 83-87, wherein the one or more bases in the first sequence read is determined to be soft-clipped by a method comprising aligning the first sequence read to a reference sequence and identifying an unaligned portion of the first sequence read.

Embodiment 89. The system of any one of claims 83-88, wherein the instructions, when executed by the one or more processors, further cause the system to:
receive a second sequence read obtained by sequencing the second strand of the cell-free DNA duplex, and
determine one or more bases in the second sequence read to be soft-clipped.

Embodiment 90. The system of claim 89, wherein the one or more bases in the second sequence read are determined to be soft-clipped according to a method comprising aligning the second sequence to a reference sequence and identifying an unaligned portion of the second sequence read.

Embodiment 91. The system of claim 89 or 90, wherein the first sequence read and the second sequence read are associated through a unique molecular identifier (UMI).

Embodiment 92. The system of claim 91, wherein the one or more bases in the first sequence read are determined to be soft-clipped according to a method comprising aligning the first sequence to the second sequence read and identifying an unaligned portion of the first sequence read.

Embodiment 93. The system of claim 91 or 92, wherein the one or more bases in the second sequence read are determined to be soft-clipped according to a method comprising aligning the second sequence to the first sequence read and identifying an unaligned portion of the second sequence read.

Embodiment 94. The system of any one of claims 89-93, wherein the second sequence read is further obtained by:
attaching a second sequencing adapter to a 3′ overhang of the second strand of a cell-free DNA duplex;
extending a 3′ end of the second sequencing adapter with inosine bases to fill the 3′ overhang of the second strand of the cell-free DNA duplex;
attaching a 3′ inosine-extended end of the second sequencing adapter to a 5′ end of a first strand of the cell-free DNA duplex, wherein the 3′ inosine-extended end provides a 5′ inosine extension of the first strand of the cell-free DNA duplex.

Embodiment 95. The system of claim 94, wherein the instructions, when executed by the one or more processors, further cause the system to:
soft clip bases corresponding to a 3′ inosine extension of the first strand of the cell-free DNA duplex from the second sequence read; and
determine a length or sequence of the 5′ overhang of the second strand of the cell-free DNA duplex based on the soft clipping of the 3′ inosine extension of the first strand of the cell-free DNA duplex.

Embodiment 96. The system of claim 94 or 95, wherein attaching the second sequencing adapter to the 3′ overhang of the second strand of a cell-free DNA duplex comprises attaching a single inosine to the 3′ overhang of the second strand of a cell-free DNA duplex and attaching the second sequencing adapter to the single inosine.

Embodiment 97. The system of claim 96, wherein the sequencing adapter comprises a 3′ cytosine overhang that complements to the single inosine attached to the 3′ overhang of the second strand of a cell-free DNA duplex.

Embodiment 98. The system of any one of claims 65-97, wherein the sequencing adapter or the second sequencing adapter comprises the UMI.

Embodiment 99. The system of any one of claims 65-98, wherein the sequencing adapter or the second sequencing adapter is a Y-shaped sequencing adapter.

Embodiment 100. The system of any one of claims 65-99, further comprising a nucleic acid amplifier configured to amplify the cell-free DNA duplex to attach a sample index to the cell-free DNA duplex.

Embodiment 101. The system of claim 100, wherein the nucleic acid amplifier is a thermal cycler.

Embodiment 102. The system of any one of claims 65-101, wherein the cell-free DNA duplex is obtained from a subject suspected of having cancer or determined to have cancer.

Embodiment 103. The system of any one of claims 65-102, wherein the cell-free DNA duplex is obtained from a subject.

Embodiment 104. The system of any one of claims 65-103, wherein the cell-free DNA duplex is obtained from a liquid biopsy sample.

Embodiment 105. The system of claim 104, wherein the sample is a liquid biopsy sample and comprises blood, plasma, cerebrospinal fluid, sputum, stool, urine, or saliva.

Embodiment 106. The system of any one of claims 65-105, wherein the cell-free DNA duplex is a circulating tumor DNA (ctDNA) duplex.

Embodiment 107. The system of any one of claims 65-106, wherein the sequencing adapter or the second sequencing adapter comprise an amplification primer binding site, a flow cell adapter sequence, or a substrate adapter sequence.

Embodiment 108. The system of any one of claims 65-107, further comprising a nucleic acid amplifier configured to amplify the first strand and the second strand of the cell-free DNA duplex.

Embodiment 109. The system of claim 108, wherein the amplifying comprises performing a polymerase chain reaction (PCR) amplification technique, a non-PCR amplification technique, or an isothermal amplification technique.

Embodiment 110. The system of any one of claims 65-109, further comprising a sequencer configured to sequence the first strand of the cell-free DNA duplex and/or the second strand of the cell-free DNA duplex.

Embodiment 111. The system of any one of claims 65-110, wherein the sequencing comprises use of a massively parallel sequencing (MPS) technique, whole genome sequencing (WGS), whole exome sequencing, targeted sequencing, direct sequencing, or Sanger sequencing technique.

Embodiment 112. The system of claim 111, wherein the sequencing comprises massively parallel sequencing, and the massively parallel sequencing technique comprises next generation sequencing (NGS).

Embodiment 113. The system of any one of claims 65-112, wherein the sequencing is performed using a next generation sequencer.

Embodiment 114. The system of any one of claims 65-113, wherein the instructions, when executed by the one or more processors, further cause the system to generate a report indicating the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex.

Embodiment 115. The system of claim 114, wherein the instructions, when executed by the one or more processors, further cause the system to transmit the report to a healthcare provider.

Embodiment 116. The system of claim 114 or 115, wherein the report is transmitted via a computer network or a peer-to-peer connection.

Embodiment 117. The system of any one of claims 65-116, wherein the instructions, when executed by the one or more processors, further cause the system to generate, by the one or more processors, a genomic profile for the subject comprising the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex.

Embodiment 118. The system of claim 117, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 119. The system of claim 117 or 118, wherein the genomic profile for the subject further comprises results from a nucleic acid sequencing-based test.

Embodiment 120. A non-transitory computer-readable storage medium storing one or more programs, the one or more programs comprising instructions, which when executed by one or more processors of a system, cause the system to:
receive a first sequence read obtained by extending a 3′ end of a first strand of a cell-free DNA duplex with inosine bases to fill a 5′ overhang of a second strand of the cell-free DNA duplex; attaching a sequencing adapter to the cell-free DNA duplex; and sequencing the first strand of the cell-free DNA duplex to generate a first sequence read;
determine one or more bases in the first sequence read to be soft-clipped;
soft clip bases corresponding to a 3′ inosine extension of the first strand of the cell-free DNA duplex from the first sequence read; and
determine a length or sequence of the 5′ overhang of the second strand of the cell-free DNA duplex based on the soft clipping of the 3′ inosine extension of the first strand of the cell-free DNA duplex.

Embodiment 121. The storage medium of claim 120, wherein the instructions further cause the system to detect a presence or absence of a disease based on the length or sequence of the 5′ overhang of the second strand of the cell-free DNA duplex.

Embodiment 122. The storage medium of claim 121, wherein the disease is a cancer.

Embodiment 123. The storage medium of any one of claims 120-122, wherein the first sequence read is further obtained by attaching a second sequencing adapter to a 3′ overhang of the second strand of the cell-free DNA duplex; extending a 3′ end of the second sequencing adapter with inosine bases to fill the 3′ overhang of the second strand of the cell-free DNA duplex; and attaching a 3′ inosine-extended end of the second sequencing adapter to a 5′ end of the first strand of cell-free DNA duplex, wherein the attached 3′ inosine-extended end provides a 5′ inosine extension of the first strand cell-free DNA duplex.

Embodiment 124. The storage medium of claim 123, wherein the instructions, when executed by the one or more processors, further cause the system to:
soft clip bases corresponding to the 5′ inosine extension of the first strand of the cell-free DNA duplex from the first sequence read; and
determine a length or sequence of the 3′ overhang of the second strand of the cell-free DNA duplex based on the soft clipping of the 5′ inosine extension of the first strand of cell-free DNA duplex.

Embodiment 125. The storage medium of claim 124, wherein the instructions further cause the system to detect a presence or absence of a disease based on the length or sequence of the 3′ overhang of the second strand of the cell-free DNA duplex.

Embodiment 126. The storage medium of any one of claims 123-125, wherein attaching the second sequencing adapter to the 3′ overhang of the second strand of the cell-free DNA duplex comprises:
extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and
attaching the second sequencing adapter to the 3′ extension of the second strand of the cell-free DNA duplex.

Embodiment 127. The storage medium of claim 126, wherein the 3′ overhang of the second strand of the cell-free DNA duplex is extended using nucleotide bases of the same base type.

Embodiment 128. The storage medium of any one of claims 120-127, wherein the one or more bases in the first sequence read is determined to be soft-clipped by a method comprising aligning the first sequence read to a reference sequence and identifying an unaligned portion of the first sequence read.

Embodiment 129. The storage medium of any one of claims 120-128, wherein the instructions, when executed by the one or more processors, further cause the system to:
receive a second sequence read obtained by sequencing the second strand of the cell-free DNA duplex, and
determine one or more bases in the second sequence read to be soft-clipped.

Embodiment 130. The storage medium of claim 129, wherein the one or more bases in the second sequence read are determined to be soft-clipped according to a method comprising aligning the second sequence to a reference sequence and identifying an unaligned portion of the second sequence read.

Embodiment 131. The storage medium of claim 129 or 130, wherein the first sequence read and the second sequence read are associated through a unique molecular identifier (UMI).

Embodiment 132. The storage medium of claim 131, wherein the one or more bases in the first sequence read are determined to be soft-clipped according to a method comprising aligning the first sequence to the second sequence read and identifying an unaligned portion of the first sequence read.

Embodiment 133. The storage medium of claim 131 or 132, wherein the one or more bases in the second sequence read are determined to be soft-clipped according to a method comprising aligning the second sequence to the first sequence read and identifying an unaligned portion of the second sequence read.

Embodiment 134. The storage medium of any one of claims 129-133, wherein the second sequence read is further obtained by extending a 3′ end of a second strand of the cell-free DNA duplex with inosine bases to fill a 5′ overhang of the first strand of the cell-free DNA duplex; and attaching the second sequencing adapter to the cell-free DNA duplex.

Embodiment 135. The storage medium of claim 134, wherein the instructions, when executed by the one or more processors, further cause the system to:
soft clip bases corresponding to a 3′ inosine extension of the second strand of the cell-free DNA duplex from the second sequence read; and
determine a length or sequence of the 5′ overhang of the first strand of the cell-free DNA duplex based on the soft clipping of the 3′ inosine extension of the second strand of the cell-free DNA duplex.

Embodiment 136. The storage medium of any one of claims 120-135, wherein extending the 3′ end of the first strand of the cell-free DNA duplex comprises forming a 3′ single inosine overhang.

Embodiment 137. The storage medium claim 136, wherein the sequencing adapter comprises a 3′ cytosine overhang that complements to the 3′ single inosine overhang.

Embodiment 138. A non-transitory computer-readable storage medium storing one or more programs, the one or more programs comprising instructions, which when executed by one or more processors of a system, cause the system to:
receive a first sequence read obtained by attaching a sequencing adapter to a 3′ overhang of a first strand of a cell-free DNA duplex; extending a 3′ end of the sequencing adapter with inosine bases to fill the 3′ overhang of the first strand of the cell-free DNA duplex; and attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex;
determine one or more bases in the first sequence read to be soft-clipped;
soft clip bases corresponding to the 5′ inosine extension of the second strand of the cell-free DNA duplex from the first sequence read; and
determine a length or sequence of the 3′ overhang of the first strand of the cell-free DNA duplex based on the soft clipping of the 5′ inosine extension of the second strand of cell-free DNA duplex.

Embodiment 139. The storage medium of claim 138, wherein the instructions further cause the system to detect a presence or absence of a disease based on the length or sequence of the 3′ overhang of the first strand of the cell-free DNA duplex.

Embodiment 140. The storage medium of claim 139, wherein the disease is cancer.

Embodiment 141. The storage medium of any one of claims 138-140, wherein attaching the second sequencing adapter to the 3′ overhang of the second strand of the cell-free DNA duplex comprises:
extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and
attaching the second sequencing adapter to the 3′ extension of the second strand of the cell-free DNA duplex.

Embodiment 142. The storage medium of claim 141, wherein the 3′ overhang of the second strand of the cell-free DNA duplex is extended using nucleotide bases of the same base type.

Embodiment 143. The storage medium of any one of claims 138-142, wherein the one or more bases in the first sequence read is determined to be soft-clipped by a method comprising aligning the first sequence read to a reference sequence and identifying an unaligned portion of the first sequence read.

Embodiment 144. The storage medium of any one of claims 138-143, wherein the instructions, when executed by the one or more processors, further cause the system to:
receive a second sequence read obtained by sequencing the second strand of the cell-free DNA duplex, and
determine one or more bases in the second sequence read to be soft-clipped.

Embodiment 145. The storage medium of claim 144, wherein the one or more bases in the second sequence read are determined to be soft-clipped according to a method comprising aligning the second sequence to a reference sequence and identifying an unaligned portion of the second sequence read.

Embodiment 146. The storage medium of claim 144 or 145, wherein the first sequence read and the second sequence read are associated through a unique molecular identifier (UMI).

Embodiment 147. The storage medium of claim 146, wherein the one or more bases in the first sequence read are determined to be soft-clipped according to a method comprising aligning the first sequence to the second sequence read and identifying an unaligned portion of the first sequence read.

Embodiment 148. The storage medium of claim 146 or 147, wherein the one or more bases in the second sequence read are determined to be soft-clipped according to a method comprising aligning the second sequence to the first sequence read and identifying an unaligned portion of the second sequence read.

Embodiment 149. The storage medium of any one of claims 144-148, wherein the second sequence read is further obtained by:
attaching a second sequencing adapter to a 3′ overhang of the second strand of a cell-free DNA duplex;
extending a 3′ end of the second sequencing adapter with inosine bases to fill the 3′ overhang of the second strand of the cell-free DNA duplex; and attaching a 3′ inosine-extended end of the second sequencing adapter to a 5′ end of a first strand of the cell-free DNA duplex, wherein the 3′ inosine-extended end provides a 5′ inosine extension of the first strand of the cell-free DNA duplex.

Embodiment 150. The storage medium of claim 149, wherein the instructions, when executed by the one or more processors, further cause the system to:
soft clip bases corresponding to a 3′ inosine extension of the first strand of the cell-free DNA duplex from the second sequence read; and
determine a length or sequence of the 5′ overhang of the second strand of the cell-free DNA duplex based on the soft clipping of the 3′ inosine extension of the first strand of the cell-free DNA duplex.

Embodiment 151. The storage medium of claim 149 or 150, wherein attaching the second sequencing adapter to the 3′ overhang of the second strand of a cell-free DNA duplex comprises attaching a single inosine to the 3′ overhang of the second strand of a cell-free DNA duplex and attaching the second sequencing adapter to the single inosine.

Embodiment 152. The storage medium of claim 151, wherein the sequencing adapter comprises a 3′ cytosine overhang that complements to the single inosine attached to the 3′ overhang of the second strand of a cell-free DNA duplex.

Embodiment 153. The storage medium of any one of claims 120-152, wherein the sequencing adapter or the second sequencing adapter comprises the UMI.

Embodiment 154. The storage medium of any one of claims 120-153, wherein the sequencing adapter or the second sequencing adapter is a Y-shaped sequencing adapter.

Embodiment 155. The storage medium of any one of claims 120-154, wherein the cell-free DNA duplex is obtained from a subject suspected of having cancer or determined to have cancer.

Embodiment 156. The storage medium of any one of claims 120-155, wherein the cell-free DNA duplex is obtained from a subject.

Embodiment 157. The storage medium of any one of claims 120-156, wherein the cell-free DNA duplex is obtained from a liquid biopsy sample.

Embodiment 158. The storage medium of claim 157, wherein the sample is a liquid biopsy sample and comprises blood, plasma, cerebrospinal fluid, sputum, stool, urine, or saliva.

Embodiment 159. The storage medium of any one of claims 120-158, wherein the cell-free DNA duplex is a circulating tumor DNA (ctDNA) duplex.

Embodiment 160. The storage medium of any one of claims 120-159, wherein the sequencing adapter or the second sequencing adapter comprise an amplification primer binding site, a flow cell adapter sequence, or a substrate adapter sequence.

Embodiment 161. The storage medium of any one of claims 120-160, wherein the sequencing comprises use of a massively parallel sequencing (MPS) technique, whole genome sequencing (WGS), whole exome sequencing, targeted sequencing, direct sequencing, or Sanger sequencing technique.

Embodiment 162. The storage medium of claim 161, wherein the sequencing comprises massively parallel sequencing, and the massively parallel sequencing technique comprises next generation sequencing (NGS).

Embodiment 163. The storage medium of any one of claims 120-162, wherein the sequencing is performed using a next generation sequencer.

Embodiment 164. The storage medium of any one of claims 120-163, wherein the instructions, when executed by the one or more processors, further cause the system to generate a report indicating the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex.

Embodiment 165. The storage medium of claim 164, wherein the instructions, when executed by the one or more processors, further cause the system to transmit the report to a healthcare provider.

Embodiment 166. The storage medium of claim 164 or 165, wherein the report is transmitted via a computer network or a peer-to-peer connection.

Embodiment 167. The storage medium of any one of claims 120-166, wherein the instructions, when executed by the one or more processors, further cause the system to generate, by the one or more processors, a genomic profile for the subject comprising the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex.

Embodiment 168. The storage medium of claim 167, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 169. The storage medium of claim 167 or 168, wherein the genomic profile for the subject further comprises results from a nucleic acid sequencing-based test.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method for determining cell-free DNA topology, comprising:
extending a 3′ end of a first strand of a cell-free DNA duplex with inosine bases to fill a 5′ overhang of a second strand of the cell-free DNA duplex;
attaching a sequencing adapter to the cell-free DNA duplex;
sequencing the first strand of the cell-free DNA duplex to generate a first sequence read;
determining, by one or more processors, one or more bases in the first sequence read to be soft-clipped;
soft clipping, by the one or more processors, bases corresponding to a 3′ inosine extension of the first strand of the cell-free DNA duplex from the first sequence read; and
determining, by the one or more processors, a length or sequence of the 5′ overhang of the second strand of the cell-free DNA duplex based on the soft clipping of the 3′ inosine extension of the first strand of the cell-free DNA duplex.

2. The method of claim 1, further comprising detecting, by the one or more processors, a presence or absence of a disease based on the length or sequence of the 5′ overhang of the second strand of the cell-free DNA duplex.

3. The method of claim 2, wherein the disease is a cancer.

4. The method of claim 1, further comprising:
attaching a second sequencing adapter to a 3′ overhang of the second strand of the cell-free DNA duplex;
extending a 3′ end of the second sequencing adapter with inosine bases to fill the 3′ overhang of the second strand of the cell-free DNA duplex;
attaching a 3′ inosine-extended end of the second sequencing adapter to a 5′ end of the first strand of cell-free DNA duplex, wherein the attached 3′ inosine-extended end provides a 5′ inosine extension of the first strand cell-free DNA duplex;
determining, by one or more processors, one or more bases attached to the 5′ end of the first sequence read to be soft-clipped;
soft clipping, by the one or more processors, bases corresponding to the 5′ inosine extension of the first strand of the cell-free DNA duplex from the first sequence read; and
determining, by the one or more processors, a length or sequence of the 3′ overhang of the second strand of the cell-free DNA duplex based on the soft clipping of the 5′ inosine extension of the first strand of cell-free DNA duplex.

5. The method of claim 4, comprising detecting, by the one or more processors, a presence or absence of a disease based on the length or sequence of 3′ overhang of the second strand of the cell-free DNA duplex.

6. The method of claim 4, wherein attaching the second sequencing adapter to the 3′ overhang of the second strand of the cell-free DNA duplex comprises:
extending the 3′ overhang of the second strand of the cell-free DNA duplex to provide a 3′ extension, wherein the second sequencing adapter comprises a 3′ overhang that complements the 3′ extension of the second strand of the cell-free DNA duplex; and
attaching the second sequencing adapter to the 3′ extension of the second strand of the cell-free DNA duplex.

7. The method of claim 1, wherein determining the one or more bases in the first sequence read to be soft-clipped comprises aligning the first sequence read to a reference sequence and identifying an unaligned portion of the first sequence read.

8. The method of claim 1, further comprising:
sequencing the second strand of the cell-free DNA duplex to generate a second sequence read; and
determining, by one or more processors, one or more bases in the second sequence read to be soft-clipped.

9. The method of claim 8, wherein determining the one or more bases in the second sequence read to be soft-clipped comprises aligning the second sequence to a reference sequence and identifying an unaligned portion of the second sequence read.

10. The method of claim 8, wherein the first sequence read and the second sequence read are associated through a unique molecular identifier (UMI).

11. The method of claim 10, wherein:
determining the one or more bases in the first sequence read to be soft-clipped comprises aligning the first sequence to the second sequence read and identifying an unaligned portion of the first sequence read; or
determining the one or more bases in the second sequence read to be soft-clipped comprises aligning the second sequence to the first sequence read and identifying an unaligned portion of the second sequence read.

12. The method of claim 8, comprising:
extending a 3′ end of a second strand of the cell-free DNA duplex with inosine bases to fill a 5′ overhang of the first strand of the cell-free DNA duplex;
attaching the second sequencing adapter to the cell-free DNA duplex;
soft clipping, by the one or more processors, bases corresponding to a 3′ inosine extension of the second strand of the cell-free DNA duplex from the second sequence read; and
determining, by the one or more processors, a length or sequence of the 5′ overhang of the first strand of the cell-free DNA duplex based on the soft clipping of the 3′ inosine extension of the second strand of the cell-free DNA duplex.

13. The method of claim 12, comprising detecting, by the one or more processors, a presence or absence of a disease based on the length or sequence of 5′ overhang of the first strand of the cell-free DNA duplex.

14. The method of claim 1, wherein extending the 3′ end of the first strand of the cell-free DNA duplex comprises forming a single 3′ inosine overhang.

15. The method of claim 14, wherein the sequencing adapter comprises a 3′ cytosine overhang that complements to the 3′ inosine overhang.

16. A method of making a sequencing construct, comprising:
attaching a sequencing adapter to a 3′ overhang of a first strand of a cell-free DNA duplex;
extending a 3′ end of the sequencing adapter with inosine bases to fill the 3′ overhang of the first strand of the cell-free DNA duplex; and
attaching a 3′ inosine-extended end of the sequencing adapter to a 5′ end of a second strand of the cell-free DNA duplex, wherein the attached 3′ inosine-extended end provides a 5′ inosine extension of the second strand of the cell-free DNA duplex.

17. The method of claim 16, wherein attaching the sequencing adapter to the 3′ overhang of the first strand of the cell-free DNA duplex comprises:
extending the 3′ overhang of the first strand of the cell-free DNA duplex to provide a 3′ extension, wherein the sequencing adapter comprises a 3′ overhang that complements the 3′ extension of the first strand of the cell-free DNA duplex; and
attaching the sequencing adapter to the 3′ extension of the first strand of the cell-free DNA duplex.

18. A method for determining cell-free DNA topology, comprising,
making the sequencing construct according to the method of claim 16;
sequencing the second strand of the cell-free DNA duplex to generate a first sequence read;
determining, by one or more processors, one or more bases in the first sequence read to be soft-clipped;
soft clipping, by the one or more processors, bases corresponding to the 5′ inosine extension of the second strand of the cell-free DNA duplex from the first sequence read; and
determining, by the one or more processors, a length or sequence of the 3′ overhang of the first strand of the cell-free DNA duplex based on the soft clipping of the 5′ inosine extension of the second strand of cell-free DNA duplex.

19. The method of claim 18, further comprising detecting, by the one or more processors, a presence or absence of disease based on the length or sequence of the 3′ overhang of the first strand of the cell-free DNA duplex.

20. The method of claim 19, wherein the disease is a cancer.
